(19) United States
(12) Patent Application Publication
(10) Pub. No.: US 2026/0308562 A1
(43) Pub. Date: Oct. 8, 2026

(54) NEW HIGH EXPRESSION AND HIGH ACTIVITY N-GLYCOSIDASES, METHODS OF PRODUCTION AND APPLICATIONS THEREOF

(71) Applicants: PARIS SCIENCES ET LETTRES, PARIS (FR); ECOLE SUPERIEURE DE PHYSIQUE ET DE CHIMIE INDUSTRIELLES DE LA VILLE DE PARIS, PARIS (FR); CENTRE NATIONAL DE LA RECHERCHE SCIENTIFIQUE (CNRS), PARIS (FR)

(72) Inventors: Joëlle VINH, Paris (FR); Yann VERDIER, Paris (FR); Zeyuan XU, Paris (FR)

(21) Appl. No.: 19/481,249

(22) PCT Filed: May 7, 2024
(86) PCT No.: PCT/EP2024/062651

§ 371 Date: Nov. 4, 2025

(30) Foreign Application Priority Data
May 9, 2023 (EP) 23315174.5

(51) Int. Cl. C12N 9/80; C12N 9/10; C12N 15/70

(52) U.S. Cl. CPC C12N 9/80; C12N 9/1088; C12N 15/70; C07K 2319/23; C12N 2800/101; C12Y 205/01018; C12Y 305/01052

(57) ABSTRACT

The present invention concerns a novel fusion polypeptide comprising a polypeptide having an N-glycosidase activity fused to an antioxidant moiety, wherein the polypeptide having an N-glycosidase activity is a polypeptide from a bacterium belonging to the Acidobacteria phylum, preferably to the Acidobacteriia class. The present invention also concerns the nucleic acid molecule, vector, and host cell encoding the fusion polypeptide, as well as compositions and kits thereof. The present invention also provides methods for purifying and/or for producing the fusion polypeptide, as well as in vitro use of the fusion polypeptide, or the nucleic acid molecule, vector, or host cell encoding the fusion polypeptide, or compositions thereof.

### TECHNICAL FIELD OF THE INVENTION

The present invention relates to the fields of enzymology and enzyme production, more specifically N-glycosidase enzymes, as well as protein analysis and deglycosylation. In particular, the invention concerns a novel fusion polypeptide comprising a polypeptide having an N-glycosidase activity fused to an antioxidant moiety, wherein the polypeptide having an N-glycosidase activity is a polypeptide from a bacterium belonging to the Acidobacteria phylum.

The present invention also concerns the nucleic acid molecule, vector, and host cell encoding the fusion polypeptide, as well as compositions and kits thereof.

The present invention also provides methods for purifying and/or for producing the fusion polypeptide, as well as in vitro use of the fusion polypeptide, or the nucleic acid molecule, vector, or host cell encoding the fusion polypeptide, or compositions thereof.

### BACKGROUND ART

N-glycosidases (also called Peptide:N-glycosidases, or peptide-N4-(N-acetyl-beta-glucosaminyl)asparagine amidases, or PNGases) are a class of enzymes that catalyzes the chemical or biochemical reaction of deglycosylation of a peptide or a polypeptide. This reaction comprises the cleavage of the link between an N-acetylglucosamine (GlcNAc) residue and an asparagine residue, from glycoproteins, glycol-polypeptides and glycopeptides. The deglycosylation reaction results in a deaminated protein or peptide and a free glycan.

Deficiencies in PNGases lead to several diseases, most of which involve the nervous system (including lysosomal storage diseases and multisystem diseases). In addition, alterations in glycosylation are often acquired in cases of cancer and inflammation, which may have important functional consequences. Indeed, N-linked glycans are able to provide structural components of cell walls and extracellular matrices, to modify protein stability and solubility, to direct the trafficking of other glycoproteins, and to mediate cell signaling (cell-cell interactions and cell-matrix interactions). N-linked (de)glycosylation plays an essential structural and functional role for various components such as antibodies, cell surfaces, and matrix proteins.

PNGases are also very useful biotechnology tools. Indeed, the removal of carbohydrates from glycoproteins may be required for numerous applications, resulting in the following advantages:
simplify the analysis of the peptide portion of a glycoprotein;
simplify the analysis of the glycan component;
remove heterogeneity in glycoproteins for X-ray crystallographic analysis;
remove carbohydrate epitopes from antigens;
enhance or reduce blood clearance rates of glycoprotein therapeutics;
investigate the role of carbohydrates in enzyme activity and solubility;
investigate ligand binding;
allow quality control of glycoprotein pharmaceuticals; etc.

In particular, several methods of protein analysis require prior deglycosylation. For instance, mass spectrometry or electrophoresis analyses are usually complicated by the presence of glycosylated peptides and polypeptides, and much less efficient than for their non-glycosylated counterpart.

Two PNGases are commercially available. Peptide:N-glycosidase F, commonly referred to as PNGase F, is an amidase of the peptide-N4-(N-acetyl-beta-glucosaminyl)asparagine amidase class. PNGase F is able to deglycosylate in the absence of denaturants, but needs extensive incubation and larger amounts of the enzyme to cleave native proteins. In addition, PNGase F cannot cleave core α1,3-fucosylated glycans from the glycoprotein.

Like PNGase F, PNGase A is an amidase having comparable properties. PNGase A mainly differs from PNGase F in that its activity is not inhibited by α1-3 Fucose residues. In addition, PNGase A has difficulty cleaving big N glycan containing sialic acid.

PNGases A and F are active in a pH range of 6-9.5. The optimal pH for these PNGases activity is around 7.5. Thus, the major limitation and drawback of these commercially available PNGases are that they are inactive at acidic pH, thereby preventing their uses in protein analysis techniques requiring acidic conditions (such as protein (including antibodies) eluted by chaotropic reagent from immune-purification).

Another limitation is that their buffer usually contains additives that are deleterious with most protein analytical techniques, including mass spectrometry.

Another PNGase has been described, PNGase H+. PNGase H+ is an N-glycosidase isolated from the bacteria Terriglobus roseus, which retains enzymatic activity in acidic conditions (Wang et al. 2014 (doi:10.1042/BSR20140148)). However, the expression level and the stability of PNGase H+ are low and incompatible with production upscaling (Guo et al., 2020 (doi: 10.3389/fbioe.2020.00741)).

Moreover, the protocol for producing and purifying this enzyme requires cell lysis conditions and buffers that are not optimal for enzyme activity and are deleterious with numerous protein analysis techniques.

Therefore, there is a need to provide novel enzymes with PNGase enzymatic activity at low pH, allowing industrially applicable production levels and PNGase activity.

### SUMMARY OF THE INVENTION

The present invention fulfills this need. Indeed, the present Inventors have designed a novel enzyme, capable of efficiently catalysing deglycosylation in extremely acidic conditions. Indeed, the Inventors demonstrate for the first time that a combination of a polypeptide having PNGase activity, with an antioxidant moiety, results in a significant increase in the PNGase expression and production yields, as well as a remarkable increase of PNGase stability and activity, not only at low pH, but also at basic pH. The Inventors have in particular developed several fusions of a polypeptide having PNGase activity (originating from acidobacteria), with a compound having antioxidant properties.

The Inventors further developed a new method for optimized PNGase production, leading to the obtention of a ready-to-use PNGase, compatible with most biotechnological applications (particularly those requiring acidic conditions), with increased long-term stability, as supported by the experimental data.

The present invention thus provides an original, stable, versatile, and efficient tool for deglycosylating polypeptides, as well as an optimized and industrially applicable production method.

The present invention thus relates to a novel fusion polypeptide comprising a polypeptide having an N-glycosidase activity fused to an antioxidant moiety, wherein the polypeptide having an N-glycosidase activity is a polypeptide from a bacterium belonging to the Acidobacteria phylum, preferably to the Acidobacteriia class.

The present invention also concerns the nucleic acid molecule, vector, and host cell encoding the fusion polypeptide, as well as compositions thereof.

The present invention also provides a novel method for purifying the fusion polypeptide, comprising the step of recovering the fusion polypeptide in a buffer comprising formic acid and/or glycine-HCl, preferably a buffer comprising formic acid.

The present invention also relates to a novel method for producing the fusion polypeptide, comprising the steps of:
a) optionally cloning the nucleic acid molecule as defined above in a vector;
b) introducing the vector of step a), or the vector as defined above, in a host cell;
c) growing the host cell of step b) under suitable conditions so as to allow the production of the fusion polypeptide; and
d) optionally, purifying the fusion polypeptide of step c) by recovering the fusion polypeptide in a buffer comprising formic acid and/or glycine-HCl, preferably a buffer comprising formic acid.

In addition, the present invention relates to a novel method for producing the fusion polypeptide, comprising the steps of:
a) optionally cloning a nucleic acid molecule encoding the polypeptide having an N-glycosidase activity as defined above, in a vector;
b) introducing the vector of step a) in a host cell;
c) growing the host cell of step b) in the presence of the antioxidant moiety, under suitable conditions so as to allow the production of a polypeptide having an N-glycosidase activity and the fusion of the antioxidant moiety to the produced polypeptide having an N-glycosidase activity; and
d) optionally, purifying the fusion polypeptide of step c) by recovering the fusion polypeptide in a buffer comprising formic acid and/or glycine-HCl, preferably a buffer comprising formic acid.

The present invention further concerns an in vitro use of the fusion polypeptide, or the nucleic acid molecule, vector, or host cell encoding the fusion polypeptide, or compositions thereof, for any biotechnological application involving deglycosylation.

The present invention also relates to a kit comprising the fusion polypeptide, or the nucleic acid molecule, vector, or host cell encoding the fusion polypeptide, or compositions thereof.

### DETAILED DESCRIPTION OF THE INVENTION

In the context of the present invention, the Inventors surprisingly found that fusion of a polypeptide having an N-glycosidase activity (in particular a polypeptide from a bacterium belonging to the Acidobacteria phylum, preferably to the Acidobacteriia class) to an antioxidant moiety (e.g., antioxidant compound) significantly increases the N-glycosidase (PNGase) activity of the polypeptide. In addition, the Inventors demonstrated that the fusion of the PNGase polypeptide to the antioxidant moiety results in a remarkable expanse of the pH range where the PNGase polypeptide is active. Indeed, the data herein disclosed reveal that the fusion polypeptide comprising a PNGase polypeptide and an antioxidant moiety has a high PNGase activity level at a broad pH range, going from basic to extremely acidic conditions (a pH range going at least from 2 to 7.5).

Through this breakthrough work, the Inventors thus provide for the first time a highly stable and versatile PNGase polypeptide, able to efficiently deglycosylate a target in both extremely acidic conditions and in basic conditions, without any loss of activity.

In particular, the data obtained by the Inventors demonstrate for the first time that the antioxidant moiety 1) improves solubilizing 2) and stabilizes the PNGase.

The data further show that PNGase expression levels are significantly increased. Indeed, the experimental results obtained by the Inventors reveal that the fusion of the PNGase polypeptide with a moiety having antioxidant properties (such as a polypeptide or a peptide having antioxidant properties) not only increases PNGase expression levels, but also facilitates the PNGase purification. Without being bound by any theory, these improvements may be due to both an improved folding and improved solubility, linked to the antioxidant properties of the moiety, and to the expression system optimized by the Inventors. Indeed, the data highlight that using expression cells comprising genes encoding rare transfer RNAs (such as a bacterial strain engineered to contain extra copies of genes that encode rare transfer RNAs) allows to further significantly increase the expression levels of the PNGase polypeptide.

Moreover, the Inventors surprisingly designed a new method of producing and/or purifying a polypeptide having an N-glycosidase activity (in particular, a polypeptide from a bacterium belonging to the Acidobacteria phylum) using a buffer containing formic acid (FA buffer) and/or glycine-HCl. The data show for the first time that eluting the PNGase polypeptide with a FA buffer (supplemented with glycine-HCl or not, or a glycine-HCl buffer) allows to significantly increase the PNGase polypeptide production and purification yield, especially for MALDI application. In addition, using a FA buffer leads to obtaining a highly pure and stable PNGase polypeptide. The PNGase polypeptide obtained with this original method is ready-to-use for any biotechnological application involving deglycosylation. Indeed, the data demonstrate that the FA buffer is compatible with any protein analysis technique, particularly those requiring acidic conditions. Moreover, the data show that the FA buffer also improves the storage of the PNGase polypeptide. Indeed, no loss of PNGase activity could be detected even after long-term (i.e., over a month) storage of the PNGase polypeptide in the FA buffer.

### Definitions

Unless specifically defined herein, all technical and scientific terms used herein have the same meaning as commonly understood by a skilled artisan in chemistry, biochemistry, cellular biology, molecular biology, and medical sciences.

As used herein throughout the entire text, the terms “a” and “an” are used in the sense that they mean “at least one”, “at least a first”, “one or more” or “one or a plurality” of the referenced compounds or steps, unless the context dictates otherwise.

The term “and/or” wherever used herein includes the meaning of “and”, “or” and “all or any other combination of the elements connected by said term”.

The term “about” or “approximately” as used herein means within 10%, preferably within 8%, and more preferably within 5%, and more preferably within 3%, and more preferably within 1% of a given value or range.

As used herein, when used to define products, compositions, cells, uses and methods, the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are open-ended and do not exclude additional, unrecited elements or method steps. Thus, a polypeptide “comprises” an amino acid sequence when the amino acid sequence might be part of the final (and/or whole) amino acid sequence of the polypeptide. Such a polypeptide can have up to several hundred additional amino acid residues (e.g., linker and antioxidant moiety as described herein). “Consisting of” means excluding any other components or steps “Consisting essentially of” means excluding other components or steps of any essential significance (however, other minor/insignificant components or steps are not excluded). In the present disclosure, the terms “comprising”, “consisting of” and “consisting essentially of” may be replaced with each other, if required.

The terms “obtained from”, “originating from”, “originate from”, or “of XXX origin” (wherein XXX is any source) are used to identify the original source of a component (e.g., polypeptide, nucleic acid molecule) but are not meant to limit the method by which the component is made which can be, for example, by chemical synthesis or recombinant means (including replacement of codon encoding amino acid residues).

The terms “polynucleotide”, “nucleic acid molecule”, and “nucleic acid” are used interchangeably herein and are understood as a polymeric or oligomeric macromolecule made from nucleotide monomers (preferably from at least 5 nucleotide monomers, also called nucleotide residues). Nucleotide monomers are composed of a nucleobase, a five-carbon sugar (such as but not limited to ribose or 2′-deoxyribose), and one to three phosphate groups. Nucleotide monomers can be chemically and/or enzymatically modified. Typically, a polynucleotide is formed through phosphodiester bonds or phosphorothioate bonds between the individual nucleotide monomers.

Nucleic acid molecules include, but are not limited to, ribonucleic acid (RNA), deoxyribonucleic acid (DNA), and mixtures thereof such as e.g., RNA-DNA hybrids (mixed polyribo-polydeoxyribonucleotides). These terms encompass single or double-stranded, linear or circular, natural or synthetic, unmodified or modified versions thereof (e.g., genetically modified polynucleotides; optimized polynucleotides), sense or antisense polynucleotides, chimeric mixture (e.g., RNA-DNA hybrids). Moreover, a polynucleotide may comprise non-naturally occurring nucleotides and may be interrupted by non-nucleotide components. Exemplary DNA nucleic acids include without limitations, complementary DNA (cDNA), genomic DNA, plasmid DNA, DNA vector, viral DNA (e.g., viral genomes, viral vectors), oligonucleotides, probes, primers, satellite DNA, microsatellite DNA, coding DNA, non-coding DNA, antisense DNA, and any mixture thereof. Exemplary RNA nucleic acids include, without limitations, messenger RNA (mRNA), precursor messenger RNA (pre-mRNA), small interfering RNA (siRNA), short hairpin RNA (shRNA), microRNA (miRNA), RNA vector, viral RNA, guide RNA (gRNA), antisense RNA, coding RNA, non-coding RNA, antisense RNA, satellite RNA, small cytoplasmic RNA, small nuclear RNA, etc. Polynucleotides described herein may be synthesized by standard methods known in the art, e.g., by use of an automated DNA synthesizer (such as those that are commercially available from Biosearch, Applied Biosystems, etc.) or obtained from a naturally occurring source (e.g., a genome, cDNA, etc.) or an artificial source (such as a commercially available library, a plasmid, etc.) using molecular biology techniques well known in the art (e.g., cloning, PCR, etc.). The nucleic acids, can e.g., be synthesized chemically, e.g., in accordance with the phosphotriester method (see, for example, Uhlmann, E. & Peyman, A. (1990) Chemical Reviews, 90, 543-584).

The terms “polypeptide” and “protein” are used interchangeably herein and refer to any peptide-bond-linked polymer of amino acids, regardless of length or post-translational modification. These terms preferably refer to polymers of amino acid residues comprising at least six amino acids covalently linked by peptide bonds. The polymer can be linear, branched, or cyclic. The polymer may comprise naturally occurring and/or amino acid analogues and it may be interrupted by non-amino acids. No limitation is placed on the maximum number of amino acids comprised in a polypeptide. As a general indication, the term refers to both short polymers (typically designated in the art as a peptide, or protein fragment) and to longer polymers (typically designated in the art as polypeptide or protein). This term encompasses native polypeptides, modified polypeptides (also designated derivatives, analogues, variants, mutants), polypeptide fragments, polypeptide multimers (e.g. dimers), mutated polypeptides, engineered polypeptides, fusion polypeptides among others. A polypeptide is understood to be any translational product of a polynucleotide regardless of size, and whether glycosylated or not, and includes peptides and proteins. Polypeptides/Proteins usable herein (including protein derivatives, protein variants, protein fragments, protein domains, protein epitopes and protein domains) can be further modified by chemical or enzymatic modification, or by physical modification (such as physical adsorption). Such a chemically modified polypeptide or enzymatically modified polypeptide comprises other chemical groups than the 20 naturally occurring amino acids. Examples of such chemical or enzymatic modifications include post-translational modifications (genetically encoded or not). Chemical or enzymatic modifications of a polypeptide may provide advantageous properties as compared to the parent polypeptide, e.g., one or more of enhanced stability, increased biological half-life, increased water solubility, increased activity, enhanced properties, labelling, etc. Such a physically modified polypeptide can be immobilized covalently or non-covalently on a support. As a general indication and without being bound therein, if the amino acid polymer contains more than 50 amino acid residues, it is preferably referred to as a polypeptide or a protein, whereas if the polymer consists of 50 or fewer amino acids, it is preferably referred to as a “peptide”. The reading and writing senses of an amino acid sequence of a polypeptide, protein and peptide as used herein are the conventional reading and writing senses. The reading and writing convention for amino acid sequences of a polypeptide, protein and peptide places the amino terminus on the left, with the sequence then being written and read from the amino terminus (N-terminus) to the carboxyl terminus (C-terminus), from left to right.

The terms “peptide” or “protein fragment” or “part of a peptide or protein” or “protein domain” herein mean a portion of a peptide or protein, i.e., a portion of the sequence of consecutive amino acids making up said peptide or protein (referred to as the peptide or protein from which the fragment is derived). When the fragment is a peptide, the fragment preferably comprises at least 6 consecutive amino acids of the peptide or protein from which it is derived; more preferably at least 8 consecutive amino acids, more preferably at least 10 consecutive amino acids, more preferably at least 12 consecutive amino acids, more preferably at least 15 consecutive amino acids, more preferably at least 20 consecutive amino acids, more preferably at least 30 consecutive amino acids of the peptide or protein from which it is derived. When the fragment is a peptide or protein fragment, the fragment preferably has a three-dimensional structure, under non-denaturing conditions (e.g., conditions that are usually non-denaturing for proteins, especially in the absence of denaturing and/or chaotropic agents). When the fragment is a peptide or protein fragment, the fragment is preferably a functional fragment. “Functional fragment” means any peptide or protein fragment, having at least one of the original functions and/or properties of the peptide or protein from which said fragment is derived. Preferably, the functional fragment performs said function with an efficiency equal to at least 30% of that of said peptide or protein or molecule, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99%, preferably at least 100% of the efficacy of said peptide or protein. Examples of protein fragments (in particular of functional fragments) include e.g., protein domains, protein epitopes, etc. Fragments usable herein (including protein fragments, peptide fragment, protein domain, protein epitopes and protein domains) can be further modified by chemical or enzymatic modification (e.g., post-translational modification(s)). When the fragment is a peptide or protein fragment, a chemically/enzymatically modified fragment comprises other chemical groups than the 20 naturally occurring amino acids (e.g., can comprise post-translational modification(s)).

The terms “amino acids”, “residues” and “amino acid residues” are used interchangeably and encompass natural amino acids as well as amino acid analogs obtained after the withdrawal of one molecule of water (e.g., non-natural, synthetic and modified amino acids, including D or L optical isomers). The terms “amino acids” are used interchangeably and encompass intact natural amino acids as well as amino acid analogs (e.g., non-natural, synthetic and modified amino acids, including D or L optical isomers).

The term “fusion” as used herein refers to the linkage (or conjugation, or bonding, or coupling) of a polypeptide to a moiety. The term “fusion polypeptide” as used herein refers to a molecule or a compound comprising a polypeptide linked (or conjugated, or bounded, or coupled) to a moiety. The terms “linkage”, “conjugation”, “bonding”, and “coupling” are herein used interchangeably. The moiety can be called “fusion partner” or “fusion moiety”.

Linkage may involve terminal coupling, inside coupling, lateral coupling, and any combination thereof. Furthermore, a fusion polypeptide of the invention may comprise one or several linked moiety(ies).

Linkage may be covalent or not. Linkage may be chemical, enzymatic, or using genetic tools.

Linkage can be carried out by any acceptable means of bonding. In this regard, linkage can thus be performed by one or more covalent, ionic, hydrogen, hydrophobic or Van der Waals bonds, cleavable or non-cleavable in physiological medium or within cells.

Furthermore, linkage can be performed at any reactive group of the polypeptide.

When the fusion is a genetic fusion, the fusion is made through the covalent linkage in a single polypeptide chain (that can be linear or not (i.e., branched) of two or more polypeptides and is performed by genetic means. In such case, linkage is made by fusing in frame the nucleic acid molecules encoding each of said polypeptides. By “fused in frame”, it is meant that the expression of the fused coding sequences results in a single polypeptide without any translational terminator between each of the fused polypeptides.

Linkage may be direct or not (i.e., via a linker). The polypeptide is said to be fused “directly” to the moiety, if there is no additional moiety or compound between the two fusion partners (the polypeptide and the moiety). For instance, the polypeptide is said to be fused directly to the moiety, if there is no additional amino acid residue between the two fusion partners (the polypeptide and the moiety). The polypeptide is said to be fused “indirectly”, or “through a linker” to the moiety, if there is a linker between the two fusion partners (the polypeptide and the moiety). Example of linkers include, but are not limited to, affinity based linkers (such as a linker derived from a couple biotin-streptavidin or from a couple antibody-antigen), and covalent linkers (such as a —SH/—NH2 group, an amine maleimide NHS-ester (such as 3-(Maleimido)propionic acid N-hydroxysuccinimide ester), or a compound targeting the C-terminal carboxyl group of a polypeptide (such as N-hydroxysuccinimide), or a peptide (such as a peptide comprising 3 to 50 (random) amino acid residues).

The linker may be a peptide or a polypeptide, for example, one comprising, or consisting essentially of, or consisting of, one or more amino acid residue(s).

When the linker is a peptide or a polypeptide, its amino acid sequence may be designed either to allow correct folding of the fusion partners (i.e., the polypeptide and the moiety) of the fusion protein (in particular allowing the fusion partners to keep their activity), and/or to allow cleavage between the two fusion partners (i.e., the polypeptide and the moiety) by a specific protease.

It is within the reach of the skilled person to assess the need to include or not a linker between the two fusion partners.

The two fusion partners (i.e., the polypeptide and the moiety) may be fused in any order, such as the moiety is N-terminal to the polypeptide (“N-terminal fusion” or “N-terminal coupling” herein means that the moiety is attached/linked/added at the N-terminus of the polypeptide, i.e. to the N-terminal amino acid residue of the polypeptide), or the moiety is C-terminal of the polypeptide (“C-terminal fusion” or “C-terminal coupling” herein means that the moiety is attached/linked/added at the C-terminus of the polypeptide, i.e. to the C-terminal amino acid residue of the polypeptide), or the moiety is within the polypeptide (“fusion within the polypeptide” or “internal fusion” or “inside coupling” herein means that the moiety is attached/linked/added within the amino acid sequence of polypeptide, and not at C-terminal extremity nor at the N-terminal extremity; i.e. the moiety is inserted between two consecutive amino acid residues of the polypeptide or the moiety substitutes one or more consecutive amino acid residues of the polypeptide), or the moiety is branched to the polypeptide (“lateral fusion” or “lateral coupling” herein means that the moiety is attached/linked/added to an amino acid residue of the polypeptide, and not at C-terminal amino acid residue nor at the N-terminal amino acid residue), and any combination thereof.

As used herein, “post-translational modification” refers to a chemical or enzymatic modification occurring naturally or not on a protein or a protein fragment, after or concomitantly to protein translation (e.g., biological or biochemical synthesis, e.g., using cellular machinery), or after or concomitantly to protein synthesis (e.g., artificial and/or chemical synthesis). This means that at least one of the naturally occurring amino acid of the protein or protein fragment is modified by the addition of at least one chemical group and/or the modification (including, but not limited to, the removal) of at least one chemical group of the naturally occurring amino acid. Examples of such chemical or enzymatic modifications include without limitation glycosylation, phosphorylation, acylation, carboxylation, acetylation, biotinylation, hydroxylation, lipoylation, amidation, ubiquitination, sumoylation, deamination, etc. By “post-translationally modified protein” it is herein referred to a protein having at least one (i.e., one or more) post-translational modification. By “post-translationally modified protein fragment” it is herein referred to a protein fragment having at least one (i.e., one or more) post-translational modification.

When referring to a protein, the term “activity”, “activity of interest” or “function” refers to any biological activity of the protein that is screened for when using the method according to the invention. Activities of interest notably include enzymatic activity, reporter activity (e.g., fluorescence activity), regulatory activity (e.g., transcription factor activity) and biological activity (e.g., drug or antibiotic activity).

“Enzymatic activity” refers to the specific catalytic activity of an enzyme.

“Enzyme” herein refers to a protein with catalytic properties (enzymatic properties). Virtually all biomolecules capable of catalysing chemical reactions in cells are enzymes; however, some catalytic biomolecules are made of RNA and are therefore distinct from enzymes: these are ribozymes. An enzyme works by lowering the activation energy of a chemical reaction, which increases the speed of the reaction. The enzyme is not modified during the reaction. The initial molecules are the substrates of the enzyme, and the molecules formed from these substrates are the products of the reaction. Enzymes are characterized by their very high specificity. Moreover, an enzyme has the characteristic of being reusable.

Enzymes are generally globular proteins that act alone or in complexes of several enzymes or subunits. Like all proteins, enzymes consist of one or more polypeptide chains folded to form a three-dimensional structure corresponding to their native state.

Enzymes are much larger molecules than their substrates. Their size can vary from about 50 residues to more than 2000 residues. Only a very small part of the enzyme—between two and four residues most often, sometimes more—is directly involved in catalysis, the so-called catalytic site (or catalytic domain). The catalytic site may be located in the vicinity of one or more binding sites, at which the substrate(s) is (are) bound and oriented to catalyse the chemical reaction. The catalytic site and the binding sites form the active site of the enzyme.

Enzymes perform a large number of functions in living organisms. For example, they can be involved in signal transduction and regulation of cellular processes, in the generation of movement, in active transmembrane transport, in digestion, in metabolism, in the immune system, in nucleic acid digestion or cleavage mechanisms or in nucleic acid production (referred to here as “nucleic acid-acting enzymes”), in prodrug conversion mechanisms (prodrug-to-drug conversion). The enzyme is preferably a prokaryotic, eukaryotic or viral enzyme, preferably an enzyme from an animal, plant, alga, microalgae, insect, microorganism, bacterium, parasite, yeast, fungus or virus, more preferably a mammalian enzyme, such as a human enzyme. The various categories of enzymes are well known to the person skilled in the art, who can refer in particular to reference works in the field (such as Schomburg D., Schomburg I., Springer Handbook of Enzymes. 2 edn. Heidelberg: Springer; 2001-2009; Liébecq C., IUPAC-IUBMB Joint Commission on Biochemical Nomenclature (JCBN) and Nomenclature Committee of IUBMB (NC-IUBMB) Biochem. Mol. Biol. Int. 1997; 43:1151-1156; IUBMB (1992), Enzyme Nomenclature 1992, Academic Press, San Diego; and specialized databases as described in Schomburg D, Schomburg I. Methods Mol Biol. 2010; 609:113-28. Enzyme databases; in particular, the BRENDA database (available, inter alia, at brenda-enzymes.org), as described, for example, by Chang A, Schomburg I, Placzek S, Jeske L, Ulbrich M, Xiao M, Sensen C W, Schomburg D, Nucleic Acids Res. 2015 January; 43. Epub 2014 Nov. 5. BRENDA in 2015: exciting developments in its 25th year of existence). It is contemplated that the term enzyme encompasses native enzymes and derivatives thereof (e.g., mutated and/or engineered enzymes), provided that such derivative is capable of having an enzymatic activity. As used herein, an “enzyme fragment” is any portion of an enzyme, preferably provided that such fragment/portion is capable of having an enzymatic activity. In the case of a protein enzyme, the enzyme fragment preferably comprises at least 6 consecutive amino acid residues of the enzyme (and is preferably an enzyme catalytic site) (preferably at least 8 consecutive amino acid residues of the enzyme, preferably at least 10, preferably at least 15, preferably at least 20, preferably at least 30 amino acid residues of the enzyme).

By “N-glycosidase” or “Peptide:N-glycosidase”, or “PNGase”, or “peptide-N4-(N-acetyl-beta-glucosaminyl)asparagine amidase”, it is herein meant a polypeptide having N-glycosidase enzymatic activity. N-glycosidase belong to a class of enzymes that catalyzes the chemical or biochemical reaction of deglycosylation of a peptide or a polypeptide. As used herein, “deglycosylation” refers to a chemical or biochemical reaction comprising the cleavage of the link between an N-acetylglucosamine (GlcNAc) residue and an asparagine residue, from glycoproteins, glycol-polypeptides and glycopeptides. The deglycosylation reaction results in a deaminated protein or peptide and a free glycan.

By “moiety” or “compound”, it is herein meant a chemical or biological entity, such as a molecule or a part/fragment thereof. The terms “moiety” and “compound” are herein considered synonyms and may be used interchangeably. Illustrative examples of moieties or compounds are, without limitation, reagents, substrates, co-substrates, cofactors, coenzymes, nucleic acid molecules (e.g., DNAs, RNAs, etc. see definition above) hormones, antigens, epitopes, ligands, antibodies, receptors, toxins, tags, and the like.

As used herein, the term “antioxidant” refers to a molecule that slows and/or prevents the oxidation of other chemicals. Oxidation is part of a redox reaction that transfers electrons from a substance to an oxidizing agent. This reaction can produce radicals that lead to destructive chain reactions. Antioxidants are able to stop these chain reactions by reducing with the radicals and thus annihilating their action. As used herein, the term “antioxidant moiety” refers to a moiety having antioxidant properties (i.e., a moiety slowing and/or preventing the oxidation of other chemicals. These terms thus include antioxidant compounds, antioxidant molecules, etc. Examples of antioxidant compounds include but are not limited to: antioxidant polypeptides, antioxidant provitamins, antioxidant vitamins (such as ascorbic acid (vitamin C), α-tocopherol (vitamin E), etc.), antioxidant flavonoids, compounds having a thiol group, compounds having a phenol group, polyphenols, carotenes, glutathione, lipoic acid, uric acid, tocopherols (such as α-tocopherol), ubiquinol (coenzyme Q), etc. Examples of antioxidant polypeptides include, but are not limited to: glutathione S-transferase, glutathione peroxidase, thioredoxin, catalase, superoxide dismutase, etc.

“Bacterium” herein means a microscopic prokaryote organism present in environment, belonging to the kingdom of bacteria. The term bacteria here designates both eubacteria and archaebacteria (or archaea). In the context of the present invention, the bacterium is preferably a bacterium belonging to the Acidobacteria phylum.

By “Acidobacteria phylum” or “Acidobacteriota phylum” it is herein referred to a branch in the classification (taxonomy) that includes Gram-negative bacteria. This phylum includes, but is not limited to, several classes of bacteria, among which: the Acidobacteriia class, the Blastocatellia class, the Holophagea class, the Polarisedimenticolia class, the Thermoanaerobaculi class, the Vicinamiibacteria class, and the Candidatus Acidiflorens. The Acidobacteria phylum is described in more details on the NCBI website (for example using the following link: “https://www.ncbi.nlm.nih.gov/Taxonomy/Browser/wwwtax.cgi?mode=Undef&id=57723&lvl=3& lin=f&keep=1&srchmode=1&unlock”)

The term “Acidobacteriia class” herein refers to a branch in the classification (taxonomy) that includes, but is not limited to, several orders of bacteria, among which: the Acidobacteriales order, the Acidoferrales order, and the Bryobacterales order.

By “Acidobacteriales order”, it is herein referred to a branch in the classification (taxonomy) that includes, but is not limited to, several families of bacteria, among which the Acidobacteriaceae family.

The term “Acidobacteriaceae family” refers to a branch in the classification (taxonomy) that includes, but is not limited to, several genera of bacteria, among which the Acidicapsa genus, the Acidipilia genus, the Bryocella genus, the Acidobacterium genus, the Bryocella genus, the Edaphobacter genus, the Silvibacterium genus, and the Terriglobus genus.

As used herein, the term “Terriglobus genus” refers to a branch in the classification (taxonomy) that includes, but is not limited to, several species of bacteria, among which the Terriglobus roseus species.

By “Terriglobus roseus”, it is herein referred to a bacterium belonging to the Terriglobus roseus species. T. roseus bacteria are aerobic Gram-negative rod lacking motility.

The terms “formic acid” or “methanoic acid” are herein used interchangeably and refer to the simplest carboxylic acid, having the chemical formula HCOOH and structure H—C(═O)—O—H.

The term “glycine” herein means an amino acid that has a single hydrogen atom as its side chain.

It is the simplest stable amino acid (carbamic acid is unstable), with the chemical formula NH2-CH2-COOH.

The term “hydrochloride acid” herein means an aqueous solution of hydrogen chloride (HCl).

By “glycine hydrochloride” or “glycine-HCl”, it is herein referred to a mixture of glycine and HCl, having the chemical formula NH2-CH2-COOH·HCl.

As used herein, “Transfer RNA” or “tRNA” refers to an adaptor molecule composed of RNA, that serves as the physical link between the mRNA and the amino acid sequence of proteins. tRNAs genes from Bacteria are typically shorter (mean=77.6 bp) than tRNAs from Archaea (mean=83.1 bp) and eukaryotes (mean=84.7 bp). The mature tRNA follows an opposite pattern with tRNAs from Bacteria being usually longer (median=77.6 nt) than tRNAs from Archaea (median=76.8 nt), with eukaryotes exhibiting the shortest mature tRNAs (median=74.5 nt).

The role of tRNA is to carry an amino acid to the protein synthesizing machinery of a cell (the ribosome). Complementation of a 3-nucleotide codon in a messenger RNA (mRNA) by a 3-nucleotide anticodon of the tRNA results in protein synthesis based on the mRNA code. The role of tRNA is thus to specify which sequence from the genetic code corresponds to which amino acid. One part of the tRNA matches the genetic code in a three-nucleotide sequence called the anticodon. The anticodon forms three complementary base pairs with a codon in mRNA during protein biosynthesis. On the end of the tRNA is a covalent attachment to the amino acid that corresponds to the anticodon sequence. Each type of tRNA molecule, determined by the anticodon sequence, can be attached to only one type of amino acid, so each organism has many types of tRNA. Because the genetic code contains multiple codons that specify the same amino acid, there are several tRNA molecules bearing different anticodons which carry the same amino acid. As used herein, “rare tRNA” refers to tRNAs corresponding to rare codons, i.e., to codons that are in short supply. For example, the rarest codons in Escherichia coli are AGG and AGA, which both encode arginine and occur only at frequencies of about 0.14% and 0.21%, respectively. The argU gene encodes the tRNA that reads both the AGA and AGG codons. Normally, this tRNA is made only in very small amounts in E. coli. If the argU gene is supplied on a plasmid (or integrated within the genome, e.g., at several copies), the host E. coli no longer has a tRNA shortage. Consequently, polypeptides or proteins (e.g., fusion polypeptide) whose genes have high levels of AGA and AGG codons are made more efficiently. Plasmids carrying all the genes for rare codon tRNAs are available commercially.

The term, “antibody” (“Ab”) is used in the broadest sense and encompasses naturally occurring antibodies and engineered antibodies; including synthetic, monoclonal, polyclonal antibodies as well as full length antibodies and fragments, variants or fusions thereof provided that such fragments, variants or fusions retain binding properties to the target protein. Such antibodies can be of any origin; human or non-human mammal (e.g., rodent or camelid antibody), or chimeric. A nonhuman antibody can be humanized by recombinant methods to reduce its immunogenicity in human. The antibody may derive from any of the well-known isotypes (e.g., IgA, IgG and IgM) and any subclasses of IgG (IgG1, IgG2, IgG3, IgG4). In addition, it may be glycosylated, partially glycosylated or non-glycosylated. Unless the context indicates otherwise, the term “antibody” also includes an antigen-binding fragment of any of the aforementioned antibodies and includes a monovalent and a divalent fragment and single chain antibodies. The term antibody also includes multi-specific (e.g., bispecific) antibody so long as it exhibits the same binding specificity as the parental antibody. It is within the skill of the artisan to screen for the binding properties of a candidate antibody.

The various categories of antibodies are well known to the person skilled in the art, who will be able to refer in particular to reference works in the field (such as Thomas D. Pollard, William C. Earnshaw, Jennifer Lippincott-Schwartz, Graham Johnson Cell Biology E-Book, Elsevier Health Sciences, 1 Nov. 2016; Mohammed Zourob, Recognition Receptors in Biosensors, DOI 10.1007/978-1-4419-0919-0, Springer-Verlag New York 2010; Abbas, Lichtman, Pillai, Cellular and Molecular Immunology E-Book, Elsevier Health Sciences, Aug. 22, 2014; Bayer V., An Overview of Monoclonal Antibodies. Semin Oncol Nurs. 2019 Sep. 2:150927; Wang W, Wang E Q, Balthasar J P. Monoclonal antibody pharmacokinetics and pharmacodynamics. Clin Pharmacol Ther. 2008 November; 84(5):548-58). For illustrative purposes, full length antibodies are glycoproteins comprising at least two heavy (H) chains and two light (L) chains inter-connected by disulfide bonds. Each heavy chain comprises a heavy chain variable region (VH) and a heavy chain constant region which is made of three CH1, CH2 and CH3 domains (optionally with a hinge between CH1 and CH2). Each light chain comprises a light chain variable region (VL) and a light chain constant region which comprises one CL domain. The VH and VL regions comprise three hypervariable regions, named complementarity determining regions (CDR), interspersed with four conserved regions named framework regions (FR) in the following order: FR1-CDR1-FR2-CDR2-FR3-CDR3-FR4. The CDR regions of the heavy and light chains are determinants for the binding specificity. As used herein, a “humanized antibody” refers to a non-human (e.g., murine, camel, rat, etc.) antibody whose protein sequence has been modified to increase its similarity to a human antibody (i.e., produced naturally in humans). The process of humanization is well known in the art and typically is carried out by substituting one or more residues of the FR regions to look like human immunoglobulin sequence whereas the vast majority of the residues of the variable regions (especially the CDRs) are not modified and correspond to those of a non-human immunoglobulin. A “chimeric antibody” comprises one or more element(s) of one species and one or more element(s) of another species, for example, a non-human antibody comprising at least a portion of a constant region (Fc) of a human immunoglobulin.

The antibody is preferably a monoclonal antibody, preferably humanized or chimeric.

Representative examples of antibody fragments and/or regions are known in the art, including heavy (H) chain, light (L), heavy chain variable region (VH), heavy chain constant region, CH domain, light chain variable region (VL), light chain constant region, CL domain, complementarity determining regions (CDR), constant region (Fc), Fab, Fab′, F(ab′)2, dAb, Fd, Fv, scFv, ds-scFv, diabody, sdAb, etc.

The term “functional antibody fragment” or “antigen-binding fragments” as used herein, refers to one or more portion or fragment of an antibody, retaining the ability to specifically bind to an antigen. Examples of binding fragments encompassed within the term “functional antibody fragment” are known in the art and include, but are not limited to, a fragment antigen binding (Fab) fragment, a Fab′ fragment, a F(ab′)₂ fragment, a heavy chain antibody, a single-domain antibody (sdAb), a single-chain fragment variable (scFv), a fragment variable (Fv), a V_{H} domain, a V_{L} domain, a single domain antibody, a scAb (single chain antibody fragment), a dAb, a Fd, a Fv, a nanobody, a minibody, an IgNAR (immunoglobulin new antigen receptor), a ds-scFv, a di-scFv, a bispecific T-cell engager (BITEs), a dual affinity re-targeting (DART) molecule, a triple body, a diabody, a single-chain diabody, an alternative scaffold protein, and any combination thereof (e.g. a fusion protein thereof).

The term “antigen” as used herein refers to any structure recognized by and/or selectively bound by molecules of the immune response, e.g., antibodies, immune cells receptors (e.g., T cell receptors (TCRs), B cell receptors (BCRs), etc.), and the like. “Antigen” herein means a natural or synthetic molecule which, when recognized by antibodies or cells of the immune system of an organism, is capable of triggering an immune response in it. Antigens are recognized by highly variable antigen receptors (such as B-cell receptor or T-cell receptor) of the adaptive immune system and may elicit a humoral or cellular immune response. Antigens that elicit such a response are also referred to as immunogens. An antigen may be foreign or toxic to the body or may be a cellular molecule (e.g., protein) that is associated with a particular disease. Antigens are usually peptides, proteins, sugars (such as polysaccharides or polyosides) and their lipid derivatives (lipids). Antigens can also be nucleic acids, or haptens (i.e., fragments of antigens). Suitable antigens include, but not limited to, biological components (e.g., peptides, polypeptides, post-translational modified polypeptides and polynucleotides); complex components (e.g., cells, cell mixtures, live or inactivated organisms such as bacteria, viruses, fungi, prions, etc.), and combinations thereof. In the case of protein antigens, the part of the antigen recognized by an antibody or a lymphocyte receptor is called an “epitope” or “antigenic determinant”. The same antigen can have several epitopes (identical or different) and thus induce a varied immune response. Antigen recognition by lymphocytes depends on the nature of the epitope. B lymphocytes bind directly to conformational epitopes through their membrane immunoglobulins. T cells recognize sequential epitopes presented by antigen-presenting cells. The antigen may be exogenous, i.e., foreign to the individual (in this case, it may be allogeneic: from an individual of the same species; or xenogeneic: from other species), or it may be endogenous, i.e., an antigen unique to the host (self-antigens). Preferred antigens for use herein are cancer/tumour antigens and antigens of pathogens (the latter preferably selected from antigens of eukaryotic or prokaryotic pathogens). The antigen is preferably a microorganism, plant, alga, microalgae, bacterium, virus, parasite, yeast, fungus, insect, animal, cancer, or tumour antigen; The antigen is preferably a protein, lipid, or sugar antigen of bacteria, virus, parasite, yeast, fungus, cancer, or tumour. The various categories of antigens are well known to the person skilled in the art, who can refer in particular to reference works in the field (such as G. J. V. Nossal, G L Ada, Antigens, Lymphoid Cells and the Immune Response, Academic Press, 1971; Marc H. V. Van Regenmortel, Structure of Antigens, Volume 3 CRC Press, Dec. 20, 1995; Edouard Drouhet, Garry T. Cole, Louis De Repentigny, Jean Latge, Fungal Antigens: Isolation, Purification, and Detection, Springer Science t Business Media, Nov. 11, 2013; Graziano D. F., Finn O. J. (2005) Tumor Antigens and Tumor Antigen Discovery. In: Khleif S. N. (eds) Tumor Immunology and Cancer Vaccines. Cancer Treatment and Research, vol 123. Springer, Boston, MA; Wang M, Claesson M H, Methods Mol Biol. 2014; 1184:309-17. Classification of human leukocyte antigen (HLA) supertypes; as well as specialized databases as described in Galperin, Fernindez-Suirez, Rigden, The 24th annual Nucleic Acids Research database issue: a look back and upcoming changes, NAR, Volume 45, Issue D1, January 2017, Pages D1-D11; in particular the PMAPP database, a database of human autoantigens (available at aagatlas.ncpsb.org, among others). It is contemplated that the term antigen encompasses native antigens and derivatives thereof (e.g., mutated and/or engineered antigens), provided that such derivative is capable of being the target of an immune response. As used herein, an “antigen fragment” is any portion of an antigen, preferably provided that such fragment/portion is capable of being the target of an immune response (e.g., epitopes, immunogenic domains, etc.). In the case of a protein antigen, the antigen fragment preferably comprises at least 6 consecutive amino acid residues of the antigen (preferably at least 8 consecutive amino acid residues of the antigen, preferably at least 10, preferably at least 15, preferably at least 20, preferably at least 30 amino acid residues of the antigen).

As used herein, “identity” or “sequence identity” means an exact sequence match between two polypeptides or amino acids, or between two nucleic acid molecules or oligonucleotides. The “percent identities” referred to in the context of the disclosure of the present invention are determined after optimal alignment of the sequences to be compared, which optimal global alignment may therefore comprise one or more insertions, deletions, truncations and/or substitutions. The alignment is global, meaning that it includes the sequences to be compared taken in their entirety over their entire length. The alignment is “optimal”, meaning that the number of insertions, deletions, truncations and/or substitutions is made as low as possible. The optimal global alignment may be performed and the percent identity may be calculated using any sequence analysis method well-known to the person skilled in the art. In addition to manual comparison, it is possible to determine global alignment using the algorithm of Needleman and Wunsch (A general method applicable to the search for similarities in the amino acid sequence of two proteins, J. Mol. Biol., 1970, March; 48(3):443-453). For nucleotide sequences, the sequence comparison may be performed using any software well-known to a person skilled in the art, such as the Needle software. The parameters used may notably be the following: “Gap open” equal to 10.0, “Gap extend” equal to 0.5, and the EDNAFULL matrix (NCBI EMBOSS Version NUC4.4). For amino acid sequences, the sequence comparison may be performed using any software well-known to a person skilled in the art, such as the Needle software. The parameters used may notably be the following: “Gap open” equal to 10.0, “Gap extend” equal to 0.5, and the BLOSUM62 matrix. Preferably, the percent identity as defined in the context of the present invention is determined via the global alignment of sequences compared over their entire length. For illustrative purposes, “at least 80% identity” herein means 80%, 81%, 82%, 83%, 84%, 85%, 86%, 87%, 88%, 89%, 90%, 91%, 92%, 93%, 94%, 95%, 96%, 97%, 98%, 99% or 100% identity.

“Vector” means a vehicle, preferably a nucleic acid molecule or a viral particle, which contains the elements necessary to allow the administration, propagation and/or expression of one or more nucleic acid molecules in a host cell or organism.

From the functional viewpoint, this term encompasses vectors for maintenance (cloning vectors), vectors for expression in various host cells or organisms (expression vectors), extrachromosomal vectors (for example, multicopy plasmids) or integration vectors (for example, designed to be integrated into a host cell genome and produce additional copies of the nucleic acid molecule it contains when the host cell replicates). This term also encompasses shuttle vectors (for example, functioning in both prokaryotic and/or eukaryotic hosts) and transfer vectors (for example, for the transfer of nucleic acid molecule(s) into the genome of a host cell).

From the structural viewpoint, the vectors can be natural, synthetic or artificial genetic sources, or a combination of natural and artificial genetic elements.

Thus, in the context of the invention, the term “vector” should be understood broadly by including plasmid and viral vectors.

A “plasmid” such as used here designates a replicable DNA construction. Usually, plasmid vectors contain selection marker genes that allow host cells bearing the plasmid to be identified and/or positively or negatively selected in the presence of the compound corresponding to the selection marker. A variety of positive or negative selection marker genes are known in the art. By way of illustration, an antibiotic resistance gene can be used as a positive selection marker making it possible to select a host cell in the presence of the corresponding antibiotic.

The term “viral vector” as used here refers to a nucleic acid vector comprising at least one element of a virus genome and can be packaged in a viral particle or a viral particle. Viral vectors can be replication-competent or selective (for example, designed to replicate better or selectively in specific host cells), or genetically deactivated such that they are replication-deficient or defective.

As used herein, the term “host cell” should be understood broadly without any limitation concerning particular organization in tissue, organ, or isolated cells. “Host cell” means a cell containing at least one polypeptide according to the invention, or at least one polypeptide capable of being obtained by the production method according to the invention, or at least one vector according to the invention, or at least one nucleic acid molecule according to the invention, or at least one vector according to the invention or any mixture of these. Advantageously, the host cell is a producer cell, i.e., a cell capable of expressing the nucleic acid molecule(s) (including gene(s)) encoded by the vector according to the invention and/or of producing the vector of the invention (i.e., a cell expressing the nucleic acid molecule(s) (including gene(s)) encoded by the vector according to the invention and/or producing the vector of the invention). This term also includes cells that express the polypeptide of the invention, and cells that can be or has been the recipient of the nucleic acid molecule encoding the polypeptide of the invention (or the vector comprising said nucleic acid molecule), as well as progeny of such cells.

The host cell can be made up of a single type of cells or a group of different types of cells. The host cell can also be a hybrid cell, i.e., resulting from the fusion of at least two cells of different types.

The host cell can belong to cultured cell lines, primary cells, stem cells, proliferative cells, or dividing cells. The term “host cells” comprises prokaryotic cells, lower eukaryotic cells such as yeast cells, and other eukaryotic cells such as archaebacteria cells, fungus cells, insect cells, plant cells, algae cells, microalgae cells, parasite cells, animal cells and mammalian cells (for example, human or non-human, preferably non-human). The host cell can be a differentiated cell, a pluripotent cell, a totipotent cell, a stem cell, an induced pluripotent stem cell (iPSC), an induced totipotent stem cell, or even an embryonic cell or embryonic stem cell. In the case of an embryonic cell or embryonic stem cell, the cell is preferably a non-human cell.

The term “host cell” more broadly comprises cells which contain or have contained the nucleic acid molecule according to the invention, as well as descendants of such cells.

The host cell can be isolated, for example, or organized in tissue, organ or even a within complete organism. In the case where the host cell is a within complete organism, said organism is not human.

### Fusion Polypeptide: PNGase Polypeptide Fusion

In the context of the present invention, the Inventors surprisingly found that fusion of a polypeptide having an N-glycosidase activity (in particular a polypeptide from a bacterium belonging to the Acidobacteria phylum, preferably to the Acidobacteriia class) to an antioxidant moiety (e.g., antioxidant compound) significantly increases the N-glycosidase (PNGase) activity of the polypeptide. In addition, the Inventors demonstrated that fusion of the PNGase polypeptide to the antioxidant moiety results in a remarkable expanse of the pH range where the PNGase polypeptide is active. Indeed, the data herein disclosed reveal that the fusion polypeptide comprising a PNGase polypeptide and an antioxidant moiety has a high PNGase activity level at a broad pH range, going from basic to extremely acidic conditions (a pH range going at least from 2 to 7.5).

Through this breakthrough work, the Inventors thus provide for the first time a highly stable and versatile PNGase polypeptide, able to efficiently deglycosylate a target in both extremely acidic conditions and in basic conditions, without any loss of activity.

In particular, the data obtained by the Inventors demonstrate for the first time that the antioxidant moiety 1) improves solubilizing 2) and stabilizes the PNGase.

The data further show that PNGase expression levels are significantly increased. Indeed, the experimental results obtained by the Inventors reveal that fusion of the PNGase polypeptide with a moiety having antioxidant properties (such as a polypeptide or a peptide having antioxidant properties) not only increases PNGase expression levels, but also facilitates the PNGase purification, thus allowing to obtain elevated PNGase polypeptide amounts.

Thus, the present invention relates to a novel fusion polypeptide comprising, or consisting essentially of, or consisting of, a polypeptide having an N-glycosidase activity fused to an antioxidant moiety, wherein the polypeptide having an N-glycosidase activity is a polypeptide from a bacterium belonging to the Acidobacteria phylum. Thus, the present invention concerns a novel PNGase polypeptide fusion.

In an advantageous embodiment, the bacterium belongs to the Acidobacteriia class, preferably to the Acidobacteriales order, more preferably to the Acidobacteriaceae family, more preferably to a genus selected from the group consisting of Acidobacterium, Edaphobacter, Silvibacterium, and Terriglobus, even more preferably to the Terriglobus genus. In a particularly preferred embodiment, the bacterium is a Terriglobus roseus bacterium.

The polypeptide having an N-glycosidase activity advantageously comprises (or consists essentially of, or consists of) an amino acid sequence having at least 75% identity with SEQ ID NO:1, preferably an amino acid sequence having at least 80% identity, more preferably at least 85% identity, more preferably at least 90%; more preferably at least 95% identity, with SEQ ID NO:1. In a specific embodiment, the polypeptide having an N-glycosidase activity comprises (or consists essentially of, or consists of) the amino acid sequence SEQ ID NO:1.

Advantageously, the antioxidant moiety is selected from the group consisting of an antioxidant polypeptide, an antioxidant provitamin, an antioxidant vitamin, an antioxidant flavonoid, and a polyphenol.

The data obtained by the Inventors show that fusion of the polypeptide having an N-glycosidase activity to a polypeptide having antioxidant properties results in a significant increase in the PNGase expression and production yields, as well as a remarkable increase of PNGase stability and activity, not only at low pH, but also at basic pH. The Inventors have in particular developed several fusions of a polypeptide having PNGase activity (originating from an acidobacteria), with a polypeptide having antioxidant properties.

Accordingly, the antioxidant moiety is preferably an antioxidant polypeptide, in particular selected from the group consisting of glutathione S-transferase (GST), glutathione peroxidase, thioredoxin, catalase, and superoxide dismutase, most preferably glutathione S-transferase (GST). The antioxidant polypeptide may be of any origin, including antioxidant polypeptide originating from any of the following groups: mammals, insects, plants, bacteria, and viruses. The antioxidant polypeptide preferably originates from a bacteria or from mammal (such as from human), more preferably from a bacteria.

In a specific embodiment, the antioxidant polypeptide is glutathione S-transferase (GST). The GST polypeptide advantageously comprises (or consists essentially of, or consists of) an amino acid sequence having at least 75% identity with SEQ ID NO:2, preferably an amino acid sequence having at least 80% identity, more preferably at least 85% identity, more preferably at least 90%; more preferably at least 95% identity, with SEQ ID NO:2. In a specific embodiment, the antioxidant polypeptide comprises (or consists essentially of, or consists of) the amino acid sequence SEQ ID NO:2.

In a specific embodiment, the antioxidant polypeptide is glutathione peroxidase. The glutathione peroxidase polypeptide advantageously comprises (or consists essentially of, or consists of) an amino acid sequence having at least 75% identity with a sequence selected from any of SEQ ID NO:3, SEQ ID NO:4, SEQ ID NO:5, SEQ ID NO:6, SEQ ID NO:7, SEQ ID NO:8, SEQ ID NO:9, and SEQ ID NO:10; preferably an amino acid sequence having at least 80% identity, more preferably at least 85% identity, more preferably at least 90%; more preferably at least 95% identity, with a sequence selected from any of SEQ ID NO:3, SEQ ID NO:4, SEQ ID NO:5, SEQ ID NO:6, SEQ ID NO:7, SEQ ID NO:8, SEQ ID NO:9, and SEQ ID NO:10. In a specific embodiment, the antioxidant polypeptide comprises (or consists essentially of, or consists of) the amino acid sequence SEQ ID NO:10. an amino acid sequence selected from any of SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

In a specific embodiment, the antioxidant polypeptide is thioredoxin. The thioredoxin polypeptide advantageously comprises (or consists essentially of, or consists of) an amino acid sequence having at least 75% identity with SEQ ID NO:11, preferably an amino acid sequence having at least 80% identity, more preferably at least 85% identity, more preferably at least 90%; more preferably at least 95% identity, with SEQ ID NO:11. In a specific embodiment, the antioxidant polypeptide comprises (or consists essentially of, or consists of) the amino acid sequence SEQ ID NO:11.

In a specific embodiment, the antioxidant polypeptide is catalase. The catalase polypeptide advantageously comprises (or consists essentially of, or consists of) an amino acid sequence having at least 75% identity with SEQ ID NO:12, preferably an amino acid sequence having at least 80% identity, more preferably at least 85% identity, more preferably at least 90%; more preferably at least 95% identity, with SEQ ID NO:12. In a specific embodiment, the antioxidant polypeptide comprises (or consists essentially of, or consists of) the amino acid sequence SEQ ID NO:12.

In a specific embodiment, the antioxidant polypeptide is superoxide dismutase. The superoxide dismutase polypeptide advantageously comprises (or consists essentially of, or consists of) an amino acid sequence having at least 75% identity with SEQ ID NO:13, preferably an amino acid sequence having at least 80% identity, more preferably at least 85% identity, more preferably at least 90%; more preferably at least 95% identity, with SEQ ID NO:13. In a specific embodiment, the antioxidant polypeptide comprises (or consists essentially of, or consists of) the amino acid sequence SEQ ID NO:13.

In a particularly advantageous embodiment, the fusion polypeptide comprises or consists essentially of, or consists of, (i) a polypeptide having an N-glycosidase activity which comprises (or consists essentially of, or consists of) an amino acid sequence having at least 75% identity with SEQ ID NO:1, preferably an amino acid sequence having at least 80% identity, more preferably at least 85% identity, more preferably at least 90%, more preferably at least 95% identity, with SEQ ID NO:1, still preferably which comprises (or consists essentially of, or consists of) the amino acid sequence SEQ ID NO:1, (ii) fused to an antioxidant polypeptide, as defined above, preferably which is glutathione S-transferase (GST), as defined above.

In the fusion polypeptide, the antioxidant moiety may be fused to the N-terminus of the polypeptide having an N-glycosidase activity (N-terminal fusion), or to the C-terminus of the polypeptide having an N-glycosidase activity (C-terminal fusion), or within the polypeptide having an N-glycosidase activity (internal fusion), or laterally to the polypeptide (lateral fusion), or any combination thereof. The experimental data obtained by the Inventors demonstrate that the PNGase polypeptide fusion is particularly stable and active when the moiety is fused to the N-terminus of the polypeptide having an N-glycosidase activity. Thus, the antioxidant moiety is preferably fused to the N-terminus of the polypeptide having an N-glycosidase activity.

In addition, the data demonstrate that this increased activity and stability regardless of the type of moiety fused to the PNGase polypeptide, when the moiety is fused to the N-terminus of the polypeptide.

Accordingly, the present invention also relates to a fusion protein comprising a polypeptide having N-glycosidase activity fused to a moiety, wherein the polypeptide having a N-glycosidase activity is a polypeptide from a bacterium belonging to the Acidobacteriia class, and wherein the moiety is fused to the N-terminus of the polypeptide having N-glycosidase activity, or within the polypeptide having a N-glycosidase activity. In such case, in particular when the moiety is not an antioxidant moiety, the moiety is preferably not fused to the C-terminus of the polypeptide having a N-glycosidase activity.

In one embodiment, the antioxidant moiety is fused to the polypeptide having an N-glycosidase activity directly or through a linker.

Advantageously, the linker is selected from the group consisting of an affinity based linker (such as a linker derived from a couple biotin-streptavidin or from a couple antibody-antigen), and a covalent linker (such as a —SH/—NH2 group, an amine maleimide NHS-ester (such as 3-(Maleimido)propionic acid N-hydroxysuccinimide ester), or a compound targeting the c-terminal carboxyl group of a polypeptide (such as N-Hydroxysuccinimide), or a peptide (such as a peptide comprising 3 to 50 (random) amino acid residues). When the antioxidant moiety is fused to the polypeptide having an N-glycosidase activity through a linker, the linker is preferably a peptide or a polypeptide.

In a preferred embodiment, the antioxidant moiety is fused directly to the polypeptide having an N-glycosidase activity (without a linker between the polypeptide and the antioxidant moiety).

Accordingly, in a specific embodiment, the antioxidant moiety is a GST fused directly to the polypeptide having an N-glycosidase activity.

The fusion polypeptide may comprise, or consist essentially of, or consist of, an amino acid sequence having at least 75% identity with SEQ ID NO: 14, preferably an amino acid sequence having at least 80% identity, more preferably at least 85% identity, more preferably at least 90%; more preferably at least 95% identity, with SEQ ID NO:14. In a specific embodiment, the fusion polypeptide comprises (or consists essentially of, or consists of) the amino acid sequence SEQ ID NO:14 (corresponding to a GST fused directly to the polypeptide having an N-glycosidase activity of Terriglobus roseus).

The Inventors surprisingly show for the first time that when the PNGase polypeptide is comprised (e.g., solubilized and/or eluted) in a buffer containing formic acid (supplemented with glycine-HCl or not, or a glycine-HCl buffer), the PNGase activity is remarkably elevated, and the PNGase polypeptide is significantly stable (in particular for short or mid-term storage, i.e., storage period lasting from two days up to several months). The PNGase polypeptide stored in a formic acid-containing buffer (FA buffer) is ready-to-use for any biotechnological application involving deglycosylation. Indeed, the data demonstrate that the FA buffer is compatible with any protein analysis technique, in particular techniques requiring acidic conditions. In addition, no loss of PNGase activity could be detected even after long term (i.e., over a month) storage of the PNGase polypeptide in the FA buffer.

Accordingly, in a preferred embodiment, the fusion polypeptide is provided in a buffer comprising (or consisting essentially of, or consisting of) formic acid and/or glycine-HCl, preferably a buffer comprising (or consisting essentially of, or consisting of) formic acid.

The buffer is preferably an aqueous buffer. The buffer preferably comprises from 0.01% volume/volume to 10% volume/volume formic acid, more preferably from 0.05% volume/volume to 8% volume/volume formic acid, more preferably from 0.1% volume/volume to 5% volume/volume formic acid, even more preferably the buffer contains about 2% volume/volume formic acid.

In addition, the experimental data obtained by the inventors demonstrate that supplementing the FA buffer (supplemented with glycine-HCl or not, or a glycine-HCl buffer) with a salt (such as ammonium acetate or sodium chloride, or combinations thereof) further increases the PNGase production yield and activity. Thus, in a preferred embodiment, the buffer further comprises a salt, preferably selected from ammonium acetate or sodium chloride, more preferably ammonium acetate. When supplemented with a salt, the buffer preferably comprises from 0.01% volume/volume to 10% volume/volume salt, more preferably from 0.05% volume/volume to 8% volume/volume salt, more preferably from 0.1% volume/volume to 5% volume/volume salt, even more preferably the buffer contains about 2% volume/volume salt. When supplemented with a salt, the buffer preferably contains from 1 mM to 20 mM salt, more preferably from 2 mM to 15 mM salt, more preferably from 3 mM to 10 mM salt, more preferably from 4 mM to 6 mM salt, even more preferably the buffer contains about 5 mM salt.

The buffer may comprise additional compounds, such as a compound selected from the group consisting of excipients, preservatives, carriers, solvents, diluents, adjuvants, dispersion media, coatings, antibacterial and/or antifungal agents, absorption agents, and the like, preferably compatible with any biotechnological application involving deglycosylation (preferably compatible with deglycosylation reactions).

Examples of suitable additional compounds include, but are not limited to surfactants (such as a detergent having the formula C₈H₁₇C₆H₄(OC₂H₄)₉₋₁₀OH (e.g. Triton™ X-100)), acidic compounds (such as hydrochloric acid (HCl), or acetic acid), amino acids (such as glycine), glycerol, glycine-HCl, Ethylenediaminetetraacetic acid (EDTA), etc.

Specific examples of suitable buffers are as follows:
Buffer 1: Aqueous buffer comprising from 0.01% volume/volume to 10% volume/volume formic acid;
Buffer 2: Buffer 1 supplemented with from 0.01% volume/volume to 10% volume/volume salt (such as ammonium acetate or sodium chloride, or combinations thereof), for example supplemented with from 1 mM to 20 mM salt;
Buffer 3: Buffer 1 supplemented with from 0.001% volume/volume to 10% volume/volume acid (such as HCl or acetic acid) (more preferably supplemented with from 0.005% volume/volume to 5% volume/volume acid, more preferably supplemented with from 0.01% volume/volume to 2% volume/volume acid);
Buffer 4: Buffer 2 supplemented with from 0.001% volume/volume to 10% volume/volume acid (such as HCl or acetic acid) (more preferably supplemented with from 0.005% volume/volume to 5% volume/volume acid, more preferably supplemented with from 0.01% volume/volume to 2% volume/volume acid);
Buffer 5: Buffer 1 supplemented with from 0.01% volume/volume to 10% volume/volume surfactant (more preferably supplemented with from 0.05% volume/volume to 5% volume/volume surfactant, more preferably supplemented with from 0.1% volume/volume to 2% volume/volume surfactant);
Buffer 6: Buffer 2 supplemented with from 0.01% volume/volume to 10% volume/volume surfactant (more preferably supplemented with from 0.05% volume/volume to 5% volume/volume surfactant, more preferably supplemented with from 0.1% volume/volume to 2% volume/volume surfactant);
Buffer 7: Buffer 3 supplemented with from 0.01% volume/volume to 10% volume/volume surfactant (more preferably supplemented with from 0.05% volume/volume to 5% volume/volume surfactant, more preferably supplemented with from 0.1% volume/volume to 2% volume/volume surfactant);
Buffer 8: Buffer 4 supplemented with from 0.01% volume/volume to 10% volume/volume surfactant (more preferably supplemented with from 0.05% volume/volume to 5% volume/volume surfactant, more preferably supplemented with from 0.1% volume/volume to 2% volume/volume surfactant);
Buffer 9: Buffer 1 supplemented with from 1 mM to 500 mM glycine (in particular glycine-HCl) (more preferably supplemented with from 10 mM to 300 mM glycine (in particular glycine-HCl), more preferably supplemented with from 50 mM to 100 mM glycine (in particular glycine-HCl);
Buffer 10: Buffer 2 supplemented with from 1 mM to 500 mM glycine (in particular glycine-HCl) (more preferably supplemented with from 10 mM to 300 mM glycine (in particular glycine-HCl), more preferably supplemented with from 50 mM to 100 mM glycine (in particular glycine-HCl);
Buffer 11: Buffer 3 supplemented with from 1 mM to 500 mM glycine (in particular glycine-HCl) (more preferably supplemented with from 10 mM to 300 mM glycine (in particular glycine-HCl), more preferably supplemented with from 50 mM to 100 mM glycine (in particular glycine-HCl);
Buffer 12: Buffer 4 supplemented with from 1 mM to 500 mM glycine (in particular glycine-HCl) (more preferably supplemented with from 10 mM to 300 mM glycine (in particular glycine-HCl), more preferably supplemented with from 50 mM to 100 mM glycine (in particular glycine-HCl);
Buffer 13: Buffer 5 supplemented with from 1 mM to 500 mM glycine (in particular glycine-HCl) (more preferably supplemented with from 10 mM to 300 mM glycine (in particular glycine-HCl), more preferably supplemented with from 50 mM to 100 mM glycine (in particular glycine-HCl);
Buffer 14: Buffer 6 supplemented with from 1 mM to 500 mM glycine (in particular glycine-HCl) (more preferably supplemented with from 10 mM to 300 mM glycine (in particular glycine-HCl), more preferably supplemented with from 50 mM to 100 mM glycine (in particular glycine-HCl);
Buffer 15: Buffer 7 supplemented with from 1 mM to 500 mM glycine (in particular glycine-HCl) (more preferably supplemented with from 10 mM to 300 mM glycine (in particular glycine-HCl), more preferably supplemented with from 50 mM to 100 mM glycine (in particular glycine-HCl);
Buffer 16: Buffer 8 supplemented with from 1 mM to 500 mM glycine (in particular glycine-HCl) (more preferably supplemented with from 10 mM to 300 mM glycine (in particular glycine-HCl), more preferably supplemented with from 50 mM to 100 mM glycine (in particular glycine-HCl);
Buffer 17: Aqueous buffer comprising from 1 mM to 500 mM glycine-HCl, more preferably from 10 mM to 300 mM glycine-HCl, more preferably from 50 mM to 100 mM glycine-HCl);
Buffer 18: Buffer 17 supplemented with from 0.01% volume/volume to 10% volume/volume salt (such as ammonium acetate or sodium chloride, or combinations thereof), for example supplemented with from 1 mM to 20 mM salt.

The fusion polypeptide as defined above may be easily obtained by standard molecular biology techniques (e.g., protein synthesis, PCR amplification, cDNA cloning, chemical synthesis) using sequence data accessible in the art and the information provided herein.

### Nucleic Acid Molecule, Vector, Host Cell, and Composition

### Nucleic Acid Molecule

The present invention also relates to a novel nucleic acid molecule encoding the fusion polypeptide as defined above, preferably wherein the antioxidant moiety is an antioxidant polypeptide.

The nucleic acid molecule encoding the fusion polypeptide as defined above, can be optimized for providing high level expression in a particular host cell or host organism.

It has been indeed observed that, when more than one codon is available to code for a given amino acid, the codon usage patterns of organisms are highly non-random (see for example Wada et al., 1992, Nucleic Acids Res. 20:2111) and the utilisation of codons may be markedly different between different hosts (see for example Nakamura et al., 1996, Nucleic Acids Res. 24:214).

For example, the antioxidant moiety may be from bacterial, viral or lower eukaryote origin and thus have an inappropriate codon usage pattern for efficient expression in higher eukaryotic cells (e.g., human), and conversely. Typically, codon optimization is performed by replacing one or more “native” (e.g., bacterial, viral or yeast) codon corresponding to a codon infrequently used in the host organism by one or more codon encoding the same amino acid which is more frequently used. It is not necessary to replace all native codons corresponding to infrequently used codons since increased expression can be achieved even with partial replacement.

Moreover, some deviations from strict adherence to optimised codon usage may be made to accommodate the introduction of restriction site(s) into the resulting nucleic acid molecule.

Further to optimization of the codon usage, expression in the host cell or host organism can further be improved through additional modifications of the recombinant nucleic sequence(s). For example, various modifications may be envisaged so as to prevent clustering of rare, non-optimal codons being present in concentrated areas and/or to suppress or modify “negative” sequence elements which are expected to negatively influence expression levels. Such negative sequence elements include without limitation the regions having very high (>80%) or very low (<30%) GC content; AT-rich or GC-rich sequence stretches; unstable direct or inverted repeat sequences; RNA secondary structures; and/or internal cryptic regulatory elements such as internal TATA-boxes, chi-sites, ribosome entry sites, and/or splicing donor/acceptor sites.

Advantageously, the nucleic acid molecule encoding the fusion polypeptide as defined above, is operably linked to suitable regulatory elements for expression in a desired host cell or host organism.

### Regulatory Elements

In accordance with the present invention, the nucleic acid molecule according to the invention may further comprise the regulatory elements necessary for the expression of the fusion polypeptide according to the invention in a host cell or host organism. The terms “regulatory elements” or “regulatory sequences” refer to any element that allows, contributes or modulates expression in a given host cell or host organism. The regulatory elements are arranged so that they function in concert for their intended purposes, for example, for a promoter to effect transcription of a nucleic acid molecule from the transcription initiation to the terminator of said nucleic acid molecule in a permissive host cell or host organism.

In a preferred embodiment, the nucleic acid molecule of the invention comprises one or more expression cassettes, each expression cassette comprising at least one promoter placed 5′ to the nucleic acid molecule (e.g., encoding the fusion polypeptide according to the invention) and one polyadenylation signal located 3′ to said nucleic acid molecule.

It will be appreciated by those skilled in the art that the choice of the regulatory sequences can depend on such factors as the nucleic acid molecule itself, the vector into which it is inserted, the recipient host cell or host organism, the level of expression desired, etc. The promoter may be of special importance. In the context of the invention, it can be constitutive directing expression of the encoded product (e.g., the fusion polypeptide according to the invention) in many types of host cells or specific to certain host cells (e.g., organ-specific regulatory sequences) or regulated in response to specific events or exogenous factors (e.g., by temperature, nutrient additive, hormone, etc.) or according to the phase of a viral cycle (e.g., late or early). One may also use promoters that are repressed during the production step in response to specific events or exogenous factors, in order to optimize vector production and circumvent potential toxicity of the expressed polypeptide(s) in the producing cells.

Various promoters may be used in the context of the present invention that are known in the state of the art. Bacterial, viral (including phage), eukaryotic (including mammal, plant, insect) promoters are particularly appropriate. Advantageously, the promoter originates from a specie belonging to the same reign as the host cell in which expression of the nucleic acid molecule of the invention is intended. For example, a bacterial promoter is advantageously selected if expression in a bacterial host cell is envisaged. Phage promoter may also be used. Representative examples of bacterial promoters include, without limitation, TAC (trp-lac hybrid promoter), Lac (lactose operon promoter), LacUV5 promotor, constitutive promoters that are derived from the E. coli lac operon, araBAD (promotor from the arabinose metabolic operon), trp (promotor from E. coli tryptophane operon), etc. Representative example of phage promoter include, without limitation T7 promoter derived from the T7 bacteriophage, T7lac (promotor from T7 bacteriophage plus lac operator), Sp6 (promotor from Sp6 bacteriophage), pL (promotor from bacteriophage lambda), T3 (promotor from T3 bacteriophage), etc.

Those skilled in the art will appreciate that the regulatory elements controlling the nucleic acid expression may further comprise additional elements for proper initiation, regulation and/or termination of transcription (e.g., a transcription termination sequences), mRNA transport (e.g., nuclear localization signal sequences, polyadenylations sequences), processing (e.g., splicing signals, self-cleaving peptides like T2A, P2A, E2A, F2A, linkers), stability (e.g., introns, like 16S/19S or chimeric human B globin/IgG, and non-coding 5′ and 3′ sequences), translation (e.g., an initiator Met, tripartite leader sequences, IRES ribosome binding sites, signal peptides, etc.), targeting sequences, linkers (e.g., linkers composed of flexible residues like glycine and serine), transport sequences, secretion signal, and sequences involved in replication or integration. Said sequences have been reported in the literature and can be readily obtained by those skilled in the art.

### Vector

The present invention also relates to a vector encoding the fusion polypeptide as defined above, such as a vector comprising at least one nucleic acid molecule as defined above (i.e., the nucleic acid molecule encoding the fusion polypeptide as defined above, preferably wherein the antioxidant moiety is an antioxidant polypeptide).

The vector may thus comprise one or more nucleic acid molecule as defined above, either identical (i.e., the vector comprises one or more identical nucleic acid molecule as defined above), or distinct (i.e., the vector comprises one or more nucleic acid molecule as defined above, each encoding a distinct fusion polypeptide, for example each encoding a distinct polypeptide having PNGase activity and/or each encoding a distinct antioxidant moiety).

The vector may be a plasmid or a viral vector.

The vector may comprise any regulatory element, preferably a (one or more) regulatory as defined above in relation to the nucleic acid molecule.

The vector may further comprise any additional nucleic acid molecule of interest (e.g., encoding a polypeptide of interest (for example a tag (e.g., a tag for immobilization, a tag for detection, etc.), a positive or negative selection marker, a reporter protein (including a fluorescent protein (such as Green fluorescent protein (GFP), Yellow fluorescent protein (YFP), Cyan fluorescent protein (CFP), Red fluorescent protein (RFP), etc.), a gene encoding a positive or negative selection marker, a reporter gene, a gene encoding a tag, etc.).

The vector as defined above, may be easily obtained by standard molecular biology techniques (e.g., PCR amplification, cDNA cloning, chemical synthesis) using sequence data accessible in the art and the information provided herein.

The nucleic acid molecule(s) (such as those encoding the fusion polypeptide comprising the nucleic acid molecule as defined above, and/or said additional nucleic acid molecule encoding a polypeptide of interest) is/are inserted into the vector by any appropriate technique known in the art.

When the vector is a viral vector, the nucleic acid molecule(s) may be inserted in any suitable location within the virus genome, e.g., within a viral gene, an intergenic region, in a non-essential gene or region or in place of viral sequences. Preference is given to insertion within the viral genome in a non-essential locus.

Insertion into the virus can be performed by routine molecular biology, e.g., as described in Sambrook et al. (2001, Molecular Cloning-A Laboratory Manual, Cold Spring Harbor Laboratory).

Insertion into a viral genome can be performed through homologous recombination as described respectively in Chartier et al. (1996, J. Virol. 70: 4805-10) and Paul et al. (2002, Cancer gene Ther. 9: 470-7).

### Host Cell

The present invention also relates to a host cell comprising the nucleic acid molecule as defined above, or the vector as defined above, or any combination thereof.

In an advantageous embodiment, the host cell has the capacity to produce/express rare codons Advantageously, the host cell comprises genes encoding one or more rare tRNAs (i.e., tRNAs corresponding to codons that are rarely expressed by the host cell (codons that are in short supply in the host cell) and/or corresponding tRNA synthetase. The host cell may be engineered to comprise extra copies of genes encoding rare tRNAs.

The host cell may be selected from prokaryotic cells, lower eukaryotic cells such as yeast cells, and other eukaryotic cells such as archaebacteria cells, fungus cells, insect cells, plant cells, algae cells, microalgae cells, parasite cells, animal cells, and mammalian cells (for example, human or non-human, preferably non-human). The host cell can be a differentiated cell, a pluripotent cell, a totipotent cell, a stem cell, an induced pluripotent stem cell (iPSC), an induced totipotent stem cell, or even an embryonic cell or embryonic stem cell. In the case of an embryonic cell or embryonic stem cell, the cell is preferably a non-human cell.

The host cell is advantageously a bacterium, preferably a bacterium of the Enterobacteriaceae family, more preferably a bacterium of the Escherichia genus, more preferably an Escherichia coli bacterium.

### Composition

The present invention also relates to a composition comprising, or consisting essentially of, or consisting of, the fusion polypeptide as defined above, the nucleic acid molecule as defined above, the vector as defined above, the host cell as defined above, or any combination thereof.

In one embodiment, the fusion polypeptide, the nucleic acid molecule, the vector, the host cell, or any combination thereof, is lyophilized. In one embodiment, the fusion polypeptide, the nucleic acid molecule, the vector, the host cell, or any combination thereof, is solubilized in a buffer.

The buffer is advantageously compatible with any biotechnological application involving deglycosylation.

The buffer preferably comprises (or consists essentially of, or consists of) formic acid. The buffer is preferably as defined above, in relation to the fusion polypeptide.

The composition may comprise additional compounds, such as a compound selected from the group consisting of excipients, preservatives, carriers, solvents, diluents, adjuvants, dispersion media, coatings, antibacterial and/or antifungal agents, absorption agents, and the like, preferably compatible with any biotechnological application involving deglycosylation (preferably compatible with deglycosylation reactions).

The additional compounds are preferably as defined above in relation to the fusion polypeptide.

### Method of Purification/Production

The Inventor unexpectedly developed a new method for optimized PNGase production, leading to obtention of a ready-to use PNGase, compatible with most biotechnological applications (particularly those requiring acidic conditions), with increased long-term stability, as supported by the experimental data. In particular, the Inventors designed a new method of producing and/or purifying a polypeptide having an N-glycosidase activity (in particular a polypeptide from a bacterium belonging to the Acidobacteria phylum, preferably to the Acidobacteriia class), using a buffer containing formic acid (FA buffer). The data show for the first time that eluting the PNGase polypeptide with a FA buffer allows to significantly increase the PNGase polypeptide production and purification yield. In addition, the use of a FA buffer leads to the obtention of a highly pure and stable PNGase polypeptide. The PNGase polypeptide obtained with this original method is ready-to-use for any biotechnological application involving deglycosylation. Indeed, the data demonstrate that the FA buffer is compatible with any protein analysis technique, in particular techniques requiring acidic conditions. Moreover, the data show that the FA buffer also improves storage of the PNGase polypeptide. Indeed, no loss of PNGase activity could be detected even after long term (i.e., over a month) storage of the PNGase polypeptide in the FA buffer.

The present invention thus provides an original, stable, versatile and efficient tool for deglycosylating polypeptides, as well as an optimized and industrially applicable production method.

The present invention thus relates to a novel method for purifying a fusion polypeptide as defined above, comprising (or consisting essentially of, or consisting of) the step of recovering the fusion polypeptide in a buffer comprising formic acid and/or glycine-HCl, preferably in a buffer comprising formic acid, preferably in a buffer comprising from 0.01% volume/volume to 10% volume/volume formic acid, more preferably from 0.05% volume/volume to 8% volume/volume formic acid, more preferably from 0.1% volume/volume to 5% volume/volume formic acid.

Advantageously, the buffer is as defined above in relation to the fusion polypeptide.

The method preferably comprises a further step of storing the fusion polypeptide directly in the buffer used for recovering the fusion polypeptide (the buffer comprising formic acid and/or glycine-HCl).

The present invention also relates to a novel method for producing a fusion polypeptide as defined above, comprising (or consisting essentially of, or consisting of) the steps of:
a) optionally cloning the nucleic acid molecule as defined above in a vector (preferably an expression vector, more preferably an expression plasmid);
b) introducing the vector of step a), or the vector as defined above, in a host cell (such as a producer cell), preferably a cell expressing rare tRNAs (i.e., a cell comprising genes encoding rare tRNA);
c) growing the host cell of step b) under suitable conditions so as to allow the production of the fusion polypeptide; and
d) optionally, purifying the fusion polypeptide of step c) by recovering the fusion polypeptide in a buffer comprising formic acid and/or glycine-HCl, preferably in a buffer comprising formic acid, preferably in a buffer comprising from 0.01% volume/volume to 10% volume/volume formic acid, more preferably from 0.05% volume/volume to 8% volume/volume formic acid, more preferably from 0.1% volume/volume to 5% volume/volume formic acid.

Advantageously, the buffer is as defined above in relation to the fusion polypeptide.

The method preferably comprises a further step, step e), of storing the fusion polypeptide of step d) directly in the buffer used for recovering the fusion polypeptide (the buffer of step d), the buffer comprising formic acid and/or glycine-HCl, preferably in a buffer comprising formic acid).

The present invention also relates to a novel method for producing a fusion polypeptide as defined above, comprising (or consisting essentially of, or consisting of) the steps of:
a) optionally cloning a nucleic acid molecule encoding the polypeptide having a N-glycosidase activity as defined above, in a vector (preferably an expression vector, more preferably an expression plasmid);
b) introducing the vector of step a) in a host cell (such as a producer cell), preferably a cell expressing rare tRNAs (i.e., a cell comprising genes encoding rare tRNA);
c) growing the host cell of step b) in the presence of an antioxidant moiety as defined above, under suitable conditions so as to allow the production of a polypeptide having a N-glycosidase activity and the fusion of the antioxidant moiety to the produced polypeptide having a N-glycosidase activity; and
d) optionally, purifying the fusion polypeptide of step c) by recovering the fusion polypeptide in a buffer comprising formic acid and/or glycine-HCl, preferably in a buffer comprising formic acid, preferably in a buffer comprising from 0.01% volume/volume to 10% volume/volume formic acid, more preferably from 0.05% volume/volume to 8% volume/volume formic acid, more preferably from 0.1% volume/volume to 5% volume/volume formic acid.

Advantageously, the buffer is as defined above in relation to the fusion polypeptide.

The method preferably comprises a further step, step e), of storing the fusion polypeptide of step d) directly in the buffer used for recovering the fusion polypeptide (the buffer of step d), the buffer comprising formic acid and/or glycine-HCl, preferably in a buffer comprising formic acid).

In an advantageous embodiment, the methods of the invention (as defined above) achieve (i.e., allow to achieve) high (elevated) expression levels of the fusion polypeptide. The methods of the invention also advantageously achieve (i.e., allow to achieve) high (elevated) yields of production and/or purification of the fusion polypeptide.

The terms “high expression levels”, “high production yield”, “high purification yield” herein means that high amounts of the fusion polypeptide are obtained (recovered) and/or that low amounts of the fusion polypeptide are lost. The terms “high amounts of the fusion polypeptide” herein mean that a net weight (dry weight) of at least 0.1 mg of fusion polypeptide is obtained (recovered), starting from a wet weight (dry weight) of 30±5 mg of host cells (i.e., the net weight of host cells after step c) as defined above). The yield may be calculated by dividing the net weight (dry weight) of fusion polypeptide obtained after step d) as defined above, by the net weight (dry weight) of host cells after step c) as defined above. Thus, “high expression levels”, “high production yield”, “high purification yield” herein means a yield superior to 1/300.

In a preferred embodiment, the yield is superior to 1/300, more preferably to 0.004, more preferably to 0.01, more preferably to 0.02, more preferably to 0.1, even more preferably to 0.2. In a preferred embodiment, the amount (net weight, dry weight) of fusion polypeptide obtained (recovered) by the methods of the invention (as defined above) is superior to 0.1 mg, preferably superior to 0.2 mg, more preferably superior to 0.3 mg, more preferably superior to 0.4 mg, more preferably superior to 0.5 mg, even more preferably superior to 1 mg.

The present invention also concerns the fusion polypeptide obtainable, or obtained, or directly obtained, by any of the methods as defined above. Such fusion polypeptide is preferably as defined above, in the section Fusion polypeptide.

### In Vitro Use

The present invention further relates to an in vitro use of the fusion polypeptide as defined above, the nucleic acid molecule as defined above, the vector as defined above, the host cell as defined above, or any combination thereof, for any biotechnological application involving deglycosylation, preferably for a biotechnological application involving deglycosylation in acidic conditions.

The biotechnological application involving deglycosylation may be selected more from the group consisting of glycosylation profiling, anti-doping control, characterization of antibodies (such as monoclonal antibodies, chimeric antibodies, humanized antibodies, phage-display produced antibodies, etc.), characterization of hormones, characterization of cellular receptors, and diagnosis and/or monitoring of a disease involving a glycosylation disorder (such as congenital disorders of glycosylation (CDG)).

Examples of glycosylation profiling applications include, but are not limited to, glycosylation profiling of glycoprotein-based drugs, (notably to verify that such glycoproteins are correctly glycosylated). Examples of glycoprotein-based drugs include, but are not limited to, those listed in Table 1 of Soli R J, Griebenow K. Glycosylation of therapeutic proteins: an effective strategy to optimize efficacy. BioDrugs. 2010 Feb. 1; 24(1):9-21. doi: 10.2165/11530550-000000000-00000. PMID: 20055529; PMCID: PMC2805475 (Table 1 of this article is thus hereby incorporated by reference in the present text).

When the biotechnological application is for diagnosing a disease involving a glycosylation disorder, the in vitro use preferably comprises the glycosylation profiling (i.e., the analyses of glycosylation) of glycoproteins in a sample obtained from a subject in need thereof (e.g., a subject susceptible to, or suspected to, suffer from a disease involving a glycosylation disorder).

When the biotechnological application is for monitoring a disease involving a glycosylation disorder, the in vitro use preferably comprises the glycosylation profiling (i.e., the analyses of glycosylation) of glycoproteins in a sample obtained from a subject in need thereof (e.g., a subject diagnosed as suffering from a disease involving a glycosylation disorder). Such monitoring may be for prognosing, diagnosing, and evaluating the efficacy of a treatment.

The present invention also relates to an in vitro method for glycosylation profiling, anti-doping control, characterization of antibodies (such as monoclonal antibodies, chimeric antibodies, humanized antibodies, phage-display produced antibodies, etc.), characterization of hormones, characterization of cellular receptors, and diagnosis and/or monitoring of a disease involving a glycosylation disorder (such as congenital disorders of glycosylation (CDG)), using the fusion polypeptide as defined above, the nucleic acid molecule as defined above, the vector as defined above, the host cell as defined above, or any combination thereof.

The in vitro method for diagnosing a disease involving a glycosylation disorder preferably comprises the step of glycosylation profiling (i.e., the analyses of glycosylation) of glycoproteins in a sample obtained from a subject in need thereof (e.g., a subject susceptible to, or suspected to, suffer from a disease involving a glycosylation disorder).

The in vitro method for monitoring a disease involving a glycosylation disorder preferably comprises the step of glycosylation profiling (i.e., the analyses of glycosylation) of glycoproteins in a sample obtained from a subject in need thereof (e.g., a subject diagnosed as suffering from a disease involving a glycosylation disorder). Such monitoring may be for evaluating the efficacy of a treatment.

### Kit

The present invention also concerns a kit comprising (or consisting essentially of, or consisting of):
a) a container comprising the fusion polypeptide as defined above, the nucleic acid molecule as defined above, the vector as defined above, the host cell as defined above, or any combination thereof (preferably wherein the fusion polypeptide, the nucleic acid molecule, the vector, the host cell, or any combination thereof, is lyophilized or solubilized in a buffer);
b) optionally a different container comprising a buffer and/or an additional compound, wherein the buffer and/or an additional compound is(are) preferably compatible with any biotechnological application involving deglycosylation; and
c) optionally instructions of use.

The buffer preferably comprises formic acid, preferably from 0.01% volume/volume to 10% volume/volume formic acid, more preferably from 0.05% volume/volume to 8% volume/volume formic acid, more preferably from 0.1% volume/volume to 5% volume/volume formic acid.

Advantageously, the buffer is as defined above in relation to the fusion polypeptide.

The additional compound may be selected from the group consisting of excipients, preservatives, carriers, solvents, diluents, adjuvants, dispersion media, coatings, antibacterial and/or antifungal agents, absorption agents, and the like, preferably compatible with any biotechnological application involving deglycosylation (preferably compatible with deglycosylation reactions).

The additional compound is preferably as defined above in relation to the fusion polypeptide.

### DESCRIPTION OF THE FIGURES

FIG. 1. Diagram comparing the classic protocol of GST purification, with the protocol of the present invention (“new protocol”).

FIG. 2. SDS PAGE result showing different protein samples. Lane 1 shows the negative control, i.e., a cell culture without induction. Lane 2 shows the sample “culture with 1 mM IPTG”. Lane 3 shows the supernatant of GST purification and induced culture (classical method, using the GSH elution buffer). Lane 4 shows GST purification elution by 50 mM GSH. Lane 5 shows the supernatant of GST purification of induced culture (method of the invention, using 1% formic acid as elution buffer).

FIG. 3. Tryptic digest LC-MS/MS result of the GST purification elution by classical method using rGSH buffer. Grey highlights correspond to the GST-PNGase peptides identified in the LC-MS. Amino acids 1-218 is GST tag and 219 to 787 is PNGase

FIG. 4. The concentration (pH) of the formic acid and the elution efficiency. Diamond shape dots represents elution with only formic acid. Triangles represent elution with formic acid supplemented with 5 mM ammonium acetate. Gray crosses represent elution with formic acid supplemented with 5 mM sodium chloride.

FIG. 5. GST-PNGase activity measurement by deglycosylation of RNase B. a) SDS-PAGE results of RNase B deglycosylation: NC: RNase B without deglycosylation; Lane 1 Promega PNGase F deglycosylated RNase B; Lane 2 GSH buffer eluted GST-PNGase deglycosylated RNase B at pH 2.6; Lane 3 GST-PNGase eluted with 2% FA supplement with 5 mM NaCl deglycosylated RNase B pH~2; Lane 4 GST-PNGase eluted with 2% FA supplement with 5 mM ammonium acetate deglycosylated RNase B pH~2; Lane 5 GSH buffer eluted GST-PNGase deglycosylated RNase B at pH 7.5; b) Gel quantification result of the deglycosylation ratio. Deglycosylation ratio=integrated density (deglycosylated band)/integrated density (un-deglycosylated band+deglycosylated band)

FIG. 6. MALDI MS acquisition: Spectrum of released glycans from RNase B glycoprotein by the afore mentioned PNGase, all the glycan HexNAc(2)Hex(5-9) can be discovered.

FIG. 7. GST-PNGase activity by deglycosylation of IgG heavy chain under strong reducing conditions. NC: IgG heavy chain without deglycosylation, Lane H1: deglycosylation with GST-PNGase under TCEP 5 mM, Lane H2: deglycosylation with GST-PNGase under TCEP 20 mM, Lane H3: deglycosylation with GST-PNGase under TCEP 50 mM, Lane H4: deglycosylation with GST-PNGase under TCEP 100 mM, Lane H5: deglycosylation with GST-PNGase under TCEP 250 mM, Lane H6: deglycosylation with GST-PNGase under DTT 20 mM, Lane H7: deglycosylation with GST-PNGase under DTT 50 mM, Lane RNB control: deglycosylsation positive control.

FIG. 8. PNGase (without GST tag) activity by deglycosylation of IgG heavy chain under strong reducing conditions. NC: IgG heavy chain without deglycosylation, Lane NH1: deglycosylation with PNGase under TCEP 40 mM, Lane NH2: deglycosylation with PNGase under TCEP 50 mM, Lane NH3: deglycosylation with PNGase under TCEP 100 mM, Lane NH4: deglycosylation with PNGase under TCEP 250 mM, Lane NH5: deglycosylation with PNGase under TCEP 300 mM, Lane NH6: deglycosylation with PNGase under DTT 50 mM.

FIG. 9. GST-PNGase and GST-PNGase F activities by deglycosylation of IgG heavy chain under strong reducing conditions. NC: IgG heavy chain without deglycosylation, Lane GST-PNase F: deglycosylation with GST-PNGase F under TCEP 450 mM, Lane GST-PNGase H+: deglycosylation with GST-PNGase of the invention under TCEP 450 mM, Lane PNGase F: Commercial PNGase F alone.

### EXAMPLES

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### 1. Example 1: Design of a New Method for Producing Ready-to-Use and Stable N-Glycosidase

### 1.1. Method of Producing a Recombinant Plasmid:

### 1.1.1. Materials

Genomic DNA (Gene bank CP003379.1) of Terriglobus roseus (strain DSM 18391) (Taxon ID 926566) was bought from DSMZ Leibniz-Institut.

### Primers:

PCR forward primer: 5′-Gaattc t ATG CCCCGCATCTTGTGCCGCCCT-3′ (SEQ ID NO: 15); PCR reverse primer: 5′-atactcgag GCGTTTCACCGGGCAGCCTGC-3′ (SEQ ID NO: 16). Primers were ordered from Eurogentec.

Plasmid: pGEX 4T3 plasmid was ordered from GE Health.

Polymerase: SuperFill high-fidelity DNA polymerase was ordered from ThermoFisher.

Competent cell: Oneshot Top10 bought from ThermoFisher, and BL21(DE3)RIPL cell bought from Agilent.

Amino acid sequences used in the examples are as follows:
PNGase: SEQ ID NO:1
GST: SEQ ID NO:2
GST-PNGase fusion protein: SEQ ID NO:14.

### 1.1.2. Method

Recombinant pGEX Plasmid Construction

The gene encoding PNGase enzyme (Tax id: 926566. Between 4437827-4439533) was amplified by PCR from the whole genome of the Terriglobus roseus (strain DSM 18391) using SuperFill high fidelity DNA polymerase following the supplier's protocol (ThermoFisher). A 3-step protocol was used with 30 cycles. Briefly, denaturation at 98° C. for 10 s, annealing at 60° C. for 10 s and extension at 72° C. 55 s. Besides, initial denaturation was set at 98° C. for 30 s and final extension was 72° C. for 5 mins. The primer pairs were designed from the PNGase gene and restriction sites of EcoR I and Xho I on pGEX 4T-3 vector (PCR forward primer is shown in SEQ ID NO: 15 and reverse primer is shown in (SEQ ID NO: 16, as detailed in paragraph 1.1.1 above).

The amplified PCR product was inserted into TOPO zero blunt II vector (ThermoFisher) according to the manufacturer's instruction and transformed into Oneshot TOP10 cell (ThermoFisher) to be cultured on LBK plate (LB represents Luria Broths, K represents antibiotic kanamycin). Colonies selected from the LBK plate were cultured in LB solution with kanamycin and further pelleted and purified for recombinant TOPO plasmid for DNA sequencing. The plasmids carrying PNGase sequence were further double digested with restriction enzyme EcoRI (NewEngland Biolabs) and Xho I (NewEngland Biolabs), and purified by Lonza flash gel system (Lonza) to get the purified double digested PNGase gene. Meanwhile, pGEX 4T-3 vector was double digested as well, and further dephosphorylated by FastAP (ThermoFisher). Then, the purification of the linearized pGEX 4T-3 vector is the same as mentioned above. Subsequently, the recombinant pGEX vector carrying the PNGase gene was constructed by ligating the two double-digested products with a T4 ligase (ThermoFisher). The recombinant pGEX plasmid was then transformed into the BL21(DE3)RIPL cell optimized for sequence containing rare codon and cultured on LBA plates (A represents antibiotic ampicillin).

### 1.2. Method of Expression and Purification of the Enzyme:

### Cell Culture

Colonies picked up from the LBAplate were cultured in LB broth with ampicillin to prepare the inoculation solution. Inoculation solution prepared in LBA broth at 17° C. for 24 h until OD value reached 2. The cell culture solution was subsequently prepared with LB broth inoculated with the inoculation solution prepared. When the OD value of the cell culture solution reached 0.6, cell induction was carried out by IPTG to a final concentration of 1 mM to trigger the GST-PNGase enzyme expression on the recombinant pGEX vector. This culture solution was incubated at 17° C. for 24 h until the OD>2, which gave the high expression level of the enzyme.

### GST-PNGase Enzyme Purification

Cells from the cell culture solution were pelleted at 13000×g at 4° C. for 20 mins and stored at −20° C. To perform the GST-PNGase enzyme purification, cell pellets from 1 mL cell culture solution was solubilized in 250 μl lysis buffer composed of Tris·HCl (125 mM, pH7.4), Lysozyme (1 mg/mL), NaCl (150 mM), DNase (2 μl), Triton X100 (0.1% v/v) and Roche Complete Protease Inhibitor (1×) and incubated on a rotator at 30 rpm for 30 mins at room temperature. Then, the crude cell lysate was mixed with the MagneGST™ Glutathione Particles that were equilibrated with MagneGST™ binding/wash buffer containing Na₂HPO₄ (4.2 mM), KH₂PO₄ (2 mM), NaCl (140 mM) and KCl (10 mM) and incubated for 30 mins at room temperature. The supernatant was removed from the particles, and the particles were further washed to remove non-specific bindings.

### Elution:

The GST-PNGase has to be eluted from the Glutathione Particles. It can be done as suggested by the classical Promega MagneGST™ Protein Purification System protocol using Tris HCl buffer (50 mM) with 50 mM r-GSH (r-GSH: reduced form of glutathione) inside (buffer hereafter called “rGSH buffer”).

However, this elution buffer is not optimal for GST-PNGase activity and for subsequent uses due to the following reasons:
1. the rGSH classic elution buffer is not at the optimal acidic pH of the GST-PNGase;
2. the rGSH classic elution buffer contains Tris HCl and GSH that unfavours applications such as MALDI TOF/TOF mass spectrometry analysis;
3. the GST binding ability on the enzyme GST-PNGase could be blocked by the GSH used for elution, further utilization is impossible
4. the rGSH classic elution buffer requires additional tedious step of buffer exchange to obtain a better activity and shelf-life.

A new elution protocol and a new elution buffer were thus designed, allowing to overcome these disadvantages. The Inventors showed for the first time that buffers containing formic acid (hereafter called “FA buffers”) provide optimal PNGase purification yield as well as optimal PNGase activity and optimal PNGase storage conditions while eliminating the need for the tedious step of buffer exchange. Eliminating this step is particularly advantageous because it reduces PNGase loss (caused by buffer exchange mentioned above) and saves time. Moreover, the Inventor showed that the PNGase can be directly stored in these new buffers containing formic acid without any activity loss, thereby further eliminating the buffer exchange step for storage.

A scheme comparing the classic protocol (rGSH protocol) with the newly designed protocol (FA protocol) is shown in FIG. 1.

The new buffers designed by the Inventors contain formic acid in different concentrations (including 0.1%, 0.5%, 1%, 2% and 4%; as shown in Table 1 below). Two supplementary salts were added to the formic acid buffer to facilitate the eluting, that are sodium chloride (NaCl) and ammonia acetate.

TABLE 1
Acidic elution buffer conditions and their pH (FA solutions)

| Elution conditions | pH |
|---|---|
| 0.1% formic acid | 2.7 |
| 0.5% formic acid | 2.3 |
| 1% formic acid | 2.2 |
| 2% formic acid | 2 |
| 4% formic acid | 1.9 |

GST-PNGase purity and sequence were estimated by SDS PAGE and LC-MS/MS. The concentration of the elution was estimated at UV280 nm.

### 1.3. Results

### 1.3.1. Expression

By constructing the recombinant plasmid based on a pGEX vector and selecting the expression system accordingly, optimized expression and culture conditions could be developed. The best conditions included preparing inoculation solution in LB broth and seeding the culture solution to be at an OD of about 0.6. Induction was done by 1 mM IPTG and cultured at 17° C. for 24 h. In addition, the data demonstrate that transformation into a bacterial strain engineered to contain extra copies of genes that encode rare tRNAs (such as BL21-CodonPlus competent cells, BL21(DE3)RIPL) allows to further increase significantly PNGase expression level.

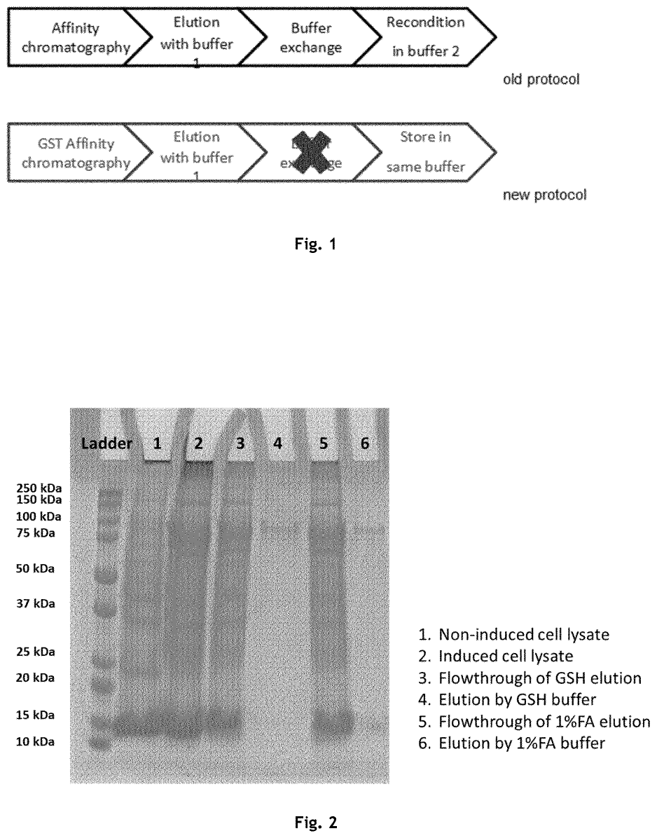

FIG. 2 presents the SDS Page result of protein expression. Comparing lanes 1 and 2 representing cultures with and without induction respectively, a strong overexpression band can be observed in lane 2 near 100 kDa, corresponding to GST-PNGase enzyme. Lane 3 is the supernatant solution of the purification process, and lane 4 is the eluted product of Tris HCl buffer (50 mM) purification with 50 mM r-GSH. This result showed that the purification was well performed as no other co-eluted protein bands appeared in the elution lane (the purity of the GST-PNGase is high). Lane 5 indicates the purification by 1% formic acid (FA) buffer.

Moreover, no degradation of the GST-PNGase in FA buffer can be observed. These data demonstrate for the first time that the GST tag, which has antioxidant properties, 1) helps solubilizing 2) and stabilizes the PNGase. The data further show that PNGase expression is significantly improved.

### 1.3.2. LC-MS/MS

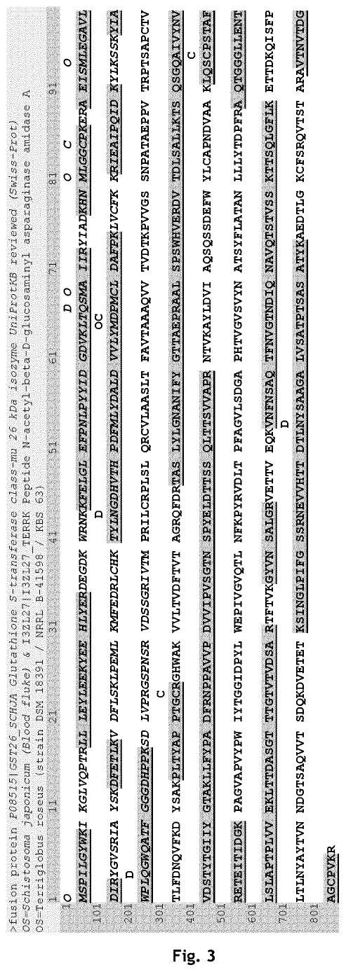

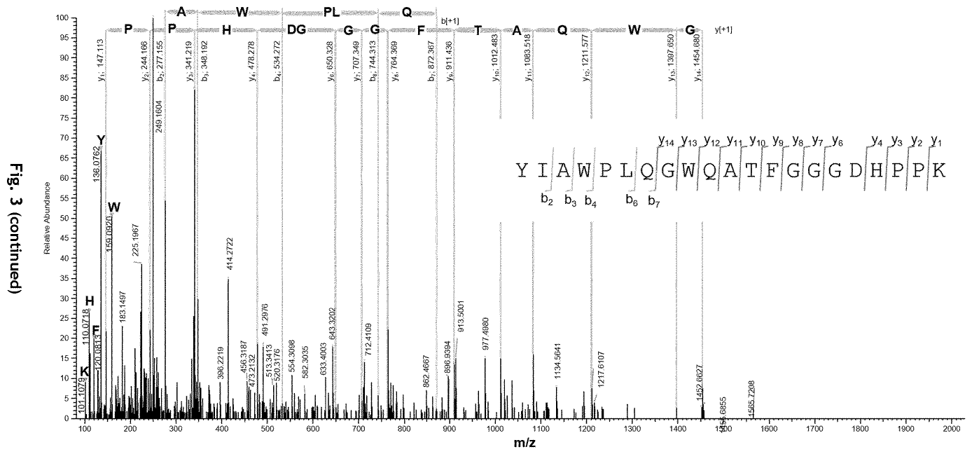

The eluted GST-PNGase was further analyzed by LC-MS/MS. From the Mascot search, the GST-PNGase protein could be identified with high score, with a protein coverage of >50% as shown in FIG. 3. Identified tryptic peptides are distributed over the entire GST-PNGase sequence. The MS/MS spectrum quality of the identified peptides from the GST-PNGase is exemplified below with a good quality. These data confirmed the successful fusion of the PNGase with the GST tag.

### 1.3.3. Concentration

2 mL culture wet pellets gave 60±10 mg wet weight (pellet wet weight estimation).

Two approaches of elution were used in this study. At the beginning, classical eluting method was utilized of GSH containing elution buffer. The Inventors were able to obtain a quantity of GST-PNGase protein of 0.1 mg after elution by GSH buffer (but before the buffer exchange step), starting from 1 mL cell culture, at a protein concentration up to 1.56±0.41 mg·mL-1, which represent a significant improvement from the previous report (Wang et al., 2014).

Thus, fusion with a moiety having antioxidant properties (here GST tag) not only facilitated the GST-PNGase purification, but also improved the expression. Without being bound by any theory, these improvements may be due both to an improved folding and solubility, linked to the antioxidant properties of GST tag, and to the expression system optimized by the Inventors. A higher enzyme concentration means a lower dilution effect during the sample processing, which is beneficial.

However, performing the buffer exchange step on the eluted GST-PNGase (aiming at removing the GSH buffer, incompatible with mass spectrometry analyses) resulted in a significant loss of GST-PNGase. After buffer exchange, the amount of GST-PNGase remaining in the recovered solution was close to 0 mg.

To overcome this drawback, the Inventors designed a new elution method by varying pH and ionic strength, with the main purpose of reducing GST-PNGase loss. The goal of the experiment was to obtain a GST-PNGase in a buffer containing as little salt as possible, while directly being at an optimal pH for the enzyme. Initial efforts trying to remove GSH in the GSH eluted GST-PNGase, by microcone spin column and Zeba destalting column, failed. In addition, this buffer exchange step led to a significant sample loss. Thus, a new protocol was designed, skipping the buffer exchange step. In this original protocol, elution was performed directly with the new formic acid buffer. No prior art describing how GSH-GST interaction could be broken by acidic conditions could be found. Only one publication by Mercado-Pimentel et al. proposed using 50 mM glycine-HCl at pH 2.5, 0.1% with addition of Triton X-100, and 150 mM NaCl to elute the GST tagged protein (Mercado-Pimentel et al., 2002). However, such a buffer is incompatible with most PNGase applications. For instance, to be compatible with MALDI matrix and mass spectrometry applications, a volatile buffer is required.

Therefore, a specialized way of eluting was designed, using newly designed formic acid buffers (FA buffers), supplemented with salts (either ammonium acetate or sodium chloride) or not.

As shown in FIG. 1, a single band between 100 and 150 kDa could be observed in lane 6, which was verified to correspond to GST-PNGase. The data demonstrate that with as little as 1% formic acid in the new elution buffer, supplemented with ammonium acetate salt, it was possible to efficiently break the interaction between GSH and GST. With the same amount of elution solution loaded onto the gel from the same pellets, a smaller band than the GSH eluted is seen. Un-bounded GST-PNGase thus could be clearly observed in lane 5, supernatant of this specialized elution approach, indicating a higher yield is limited by the capacity of the GSH beads used in the purification step. It should be mentioned here that the capacity of the beads used was not big enough to capture all the produced GST-PNGase (non negligeable amounts of PNGase were therefore trashed in the flowthrough). As a consequence, the estimated production yield (estimated on the basis of the elution yield) can be further increased using beads with higher capacity.

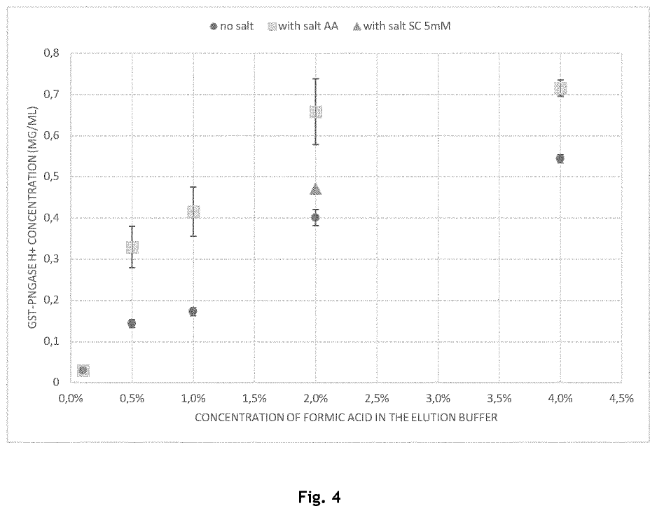

It is also possible to perform a second elution to further increase the estimated production yield. Other formic acid concentrations for elution were also tested, illustrated in FIG. 4. Lozenges represent elution with formic acid only. Triangles represent elution by formic acid supplemented with ammonium acetate. It can be observed from the lozenges that elution efficiency increases with formic acid concentration. Under same formic acid concentration, higher elution efficiency was reached by adding 5 mM ammonium acetate, which probably breaks the electrostatic interaction. Eluting with 2% formic acid, supplemented with 2% sodium chloride also resulted in higher elution efficiency than with formic acid only, though to a lesser extent than with FA buffer supplemented with ammonium acetate. Elution with 4% FA buffer supplemented with ammonium acetate salt, at least 0.14 mg GST-PNGase were eluted from a 1 mL cell culture pellet, which is significantly higher than with the GSH buffer.

Altogether, the data show that elution with FA buffers significantly increases PNGase elution yield. Supplementing FA buffers with a salt (either ammonium acetate or sodium chloride) further increased the yield (especially with ammonium acetate), as well as PNGase activity. Without being bound by any theory, it is believed that ammonium acetate or sodium chloride acts by breaking the electrostatic interactions, thereby further increasing elution yields.

### 2. Example 2: GST-PNGase is High Stable and Active at a Broad pH Range

### 2.1. Activity of the GST-PNG

### 2.1.1. On Gel

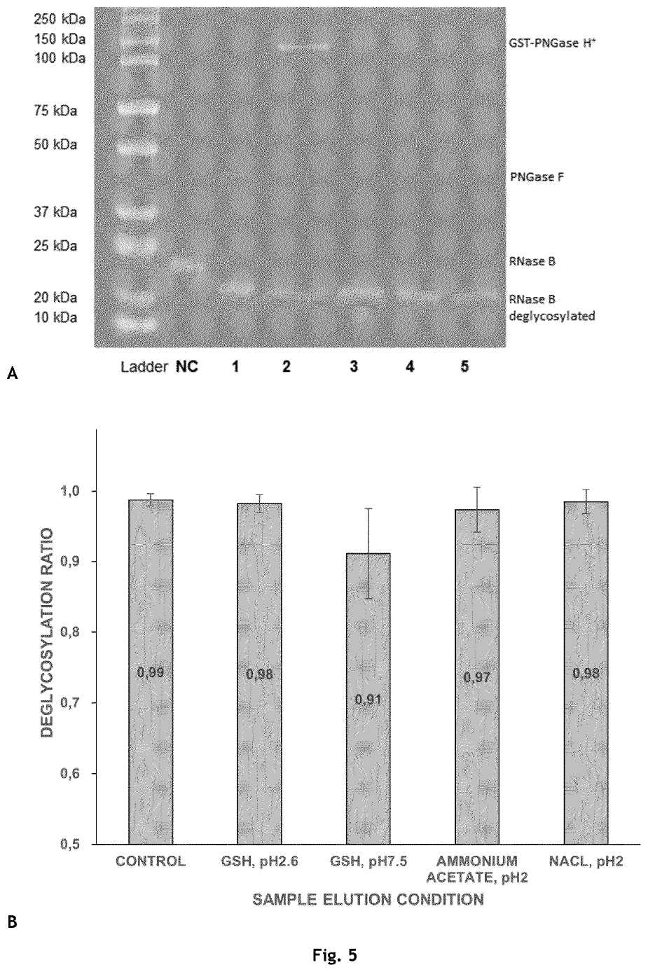

Deglycosylation of RNase B was tested and the results can be seen in FIG. 5a. Deglycosylation performance of GST-PNGase eluted in the classical manner with rGSH buffer was compared in both acidic (pH 2.6) and in basic conditions (pH 7.5), with a same enzyme substrate ratio of 3.3. The results are shown in lane 2 and 5 of FIG. 5a). Compared with the undeglycosylated RNase B in lane NC, a clear mass shift was observed for both acidic and basic deglycosylation conditions, which is the same band given by PNGase F deglycosylated in lane 1. This result shows that GST-PNGase is able to work in a broad pH range. The significant loss of activity observed for the original PNGase H+ at pH larger than 3.5 and almost goes to 0 when pH larger than 6 (as described by Wang et al., 2014) was not seen here. Thus, the data demonstrate that fusion of PNGase with GST tag significantly increases the pH range of activity. Signals in lanes 2 and 3 of FIG. 5a) show the deglycosylation result by the GST-PNGase eluted with 2% formic acid with NaCl and ammonium acetate as addition salt. The enzyme substrate ratio is calculated to be 0.9 and 1.2 respectively. Both of the samples give a deglycosylation band close to 20 kDa.

Thus, the deglycosylation efficiency could be estimated by gel quantification of triplicated experiments. And final results are presented in FIG. 5b). From the bar chart, GST-PNGase achieved a full deglycosylation of RNase B, under acidic conditions. No difference was observed between GST-PNGase eluted classically (rGSH buffer) or with FA buffer. Under basic conditions of pH 7.5, GST-PNGase retained 90% activity. Previous work reported that PNGase in 500 mM (around 2%) formic acid led to tremendous activity loss, which was not the case with GST-PNGase. A high deglycosylation ratio was also obtained with PNGase obtained by elution with 2% formic acid supplemented with 5 mM sodium chloride, though slightly lower than with PNGase obtained by elution with 2% formic acid supplemented with ammonium acetate.

Altogether, the data reveal that GST-PNGase is highly active at a broad pH range, going from basic to extremely acidic conditions (a pH range going at least from 2 to 7.5). Through this breakthrough work, the Inventors thus provide for the first time a highly stable and versatile PNGase, able to efficiently deglycosylate a target in both extremely acidic conditions and in basic conditions, without loss of activity.

### 2.1.2. MALDI Mass Spectrometry Acquisition

Deglycosylation of RNase B by GST-PNGase in 2% of formic acid and the released glycan spectra was acquired with MALDI MS using 3-aminoquinolin (3AQ) as MALDI matrix, shown in FIG. 6. The GST-PNGase activity can be discovered and buffer compatibility to the MS acquisition were proved by all the five glycans from RNase B glycoproteins were able to be detected on the spectra, which are in the sodium adduct form with 3AQ labeled at the reducing end.

### 2.2. Storage

Stability of PNGase was compared after storage in the classic buffer (rGSH) and after storage in the FA buffer. The data demonstrate that PNGase activity is dramatically reduced (even lost) after one-week storage in storage in classic buffer (rGSH), whereas no activity loss was observed after one month storage in FA buffer (data not shown).

### 3. Example 3: GST-PNGase Remains Active Under Strong Reducing Environment

Activity of the GST-PNGase was tested on a pharmaceutically important glycoprotein Immunoglobulin G (IgG), under strong reducing environment.

### 3.1. Experimental Conditions

Glycoprotein IgG was used as a deglycosylation target. It comprises a glycosylated heavy chain at around 50 kDa and a light chain of around 25 kDa without glycosylation. Deglycosylation of the IgG heavy chain was thus assessed.

The protein IgG I4505 was bought from Merck (Darmstadt, Germany). 10 μg of IgG was used for each condition.

IgG was first reduced under different strong reducing environments:
Denaturation environment 1: Tris(2-carboxyethyl)phosphine (TCEP) at 5 mM, 20 mM, 50 mM, 100 mM or 250 mM, for 1 h at room temperature on thermomixer at 750 rpm;
Denaturation environment 2: Dithiothreitol (DTT) at 20 mM or 50 mM, for 30 min at 56° C. on thermomixer at 750 rpm.

Deglycosylation assay was thus performed under the optimal condition of GST-PNGase (i.e. at 22° C. at pH=2.5) for overnight reaction on thermomixer at 750 rpm, while the reducing conditions were kept the same concentrations as mentioned above.

Deglycosylated products were analyzed with SDS-PAGE gel stained by Coomassie Blue.

### 3.2. Results

The activity of GST-PNGase was tested with two different reducing conditions created by the strong reducing reagents DTT and TCEP, where both reagents are stronger reducing reagents than formic acid (FA).

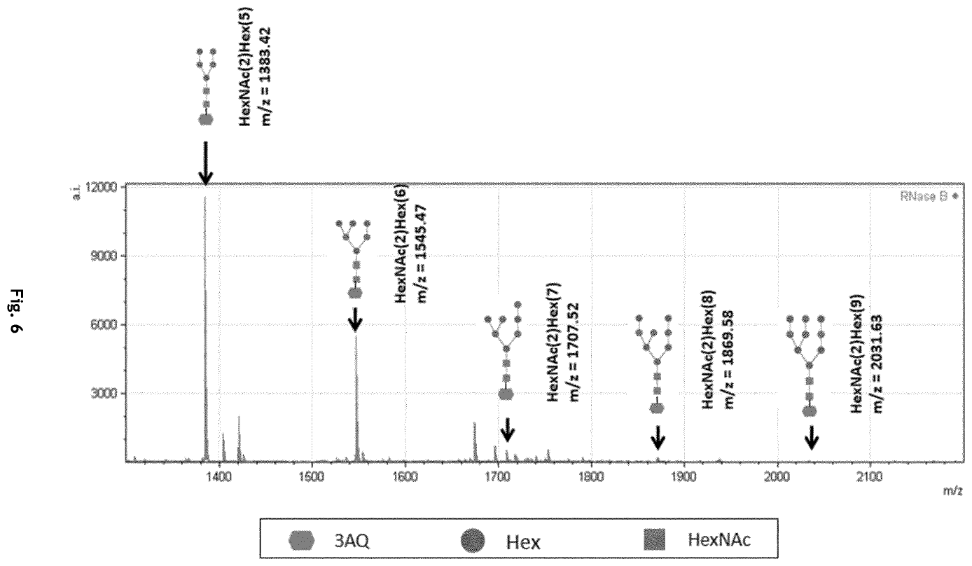

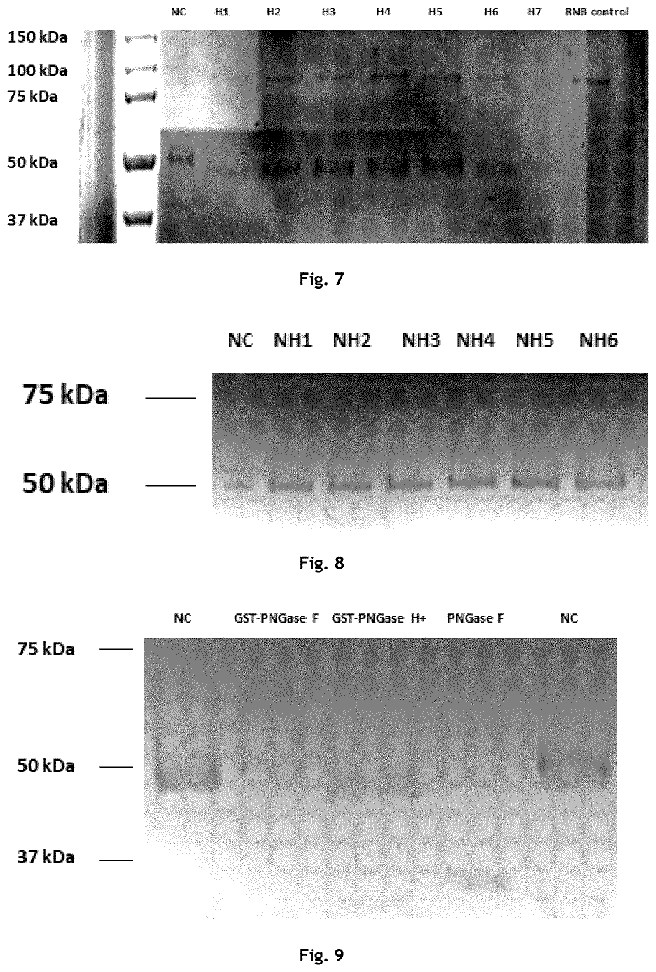

The gel displayed on FIG. 7 shows the deglycosylation experiments results. Lanes H1-H7 represent the above-defined experimental conditions, namely H1: TCEP 5 mM, H2: TCEP 20 mM, H3: TCEP 50 mM, H4: TCEP 100 mM, H5: TCEP 250 mM, H6: DTT 20 mM and H7: DTT 50 mM. NC represents a negative control where no N-glycosidase was added to the sample thus IgG heavy chain was not deglycosylated. A RNB control was used to measure the activity of the GST-PNGase to ensure a positive enzymatic activity maintained during the reaction. Protein ladder was used and indicated by the left side of the gel to indicate the bands of interest.

From the gel, bands representing GST-PNGase between 75-100 kDa can be observed. By the negative control, undeglycosylated IgG heavy chain band is observed at around 50 kDa in lane NC. Compared to this control, it can be seen that IgG heavy chain mass shifted after the deglycosylation assay in lanes H1 to H7 with all the experimental conditions, where the reactions were reduced by TCEP and DTT, indicating the GST-PNGase enzymatic activity remained in such strong reducing environment.

### 4. Example 4: PNGase without GST Tag is No Longer Active Under Strong Reducing Environment

Activity of the PNGase without GST tag was tested on a pharmaceutically important glycoprotein Immunoglobulin G (IgG), under strong reducing environment.

### 4.1. Experimental Conditions

### GST Tag Removal

Thrombin T4648 purchased from Merck (Darmstadt, Germany) was used to remove the GST tag from the expressed GST-PNGase.

The experiment was performed under a 1:100 (1 unit thrombin to 100 μg protein IgG) ratio at room temperature (22° C.) for overnight (15 h30 min) reaction on a thermomixer at 650 rpm.

The resulting PNGase was used for subsequent deglycosylation experiments.

### Deglycosylation

Glycoprotein IgG was used as a deglycosylation target. It comprises a glycosylated heavy chain at around 50 kDa and a light chain of around 25 kDa without glycosylation. Deglycosylation of the IgG heavy chain was thus assessed.

The protein IgG I4505 was bought from Merck (Darmstadt, Germany). 10 μg of IgG was used for each condition.

IgG was first reduced under different strong reducing environments:
Denaturation environment 1: Tris(2-carboxyethyl)phosphine (TCEP) at 40 mM, 50 mM, 100 mM, 250 mM or 300 mM, for 1 h at room temperature on thermomixer at 750 rpm;
Denaturation environment 2: DTT at 50 mM, for 30 min at 56° C. on thermomixer at 750 rpm.

Deglycosylation assay was then performed under the optimal condition of PNGase (i.e., at 22° C. at pH=2.5) for overnight reaction on thermomixer at 650 rpm, while the reducing conditions were kept the same concentrations as mentioned above.

Deglycosylated products were analyzed with SDS-PAGE gel stained by Coomassie Blue.

### 4.2. Results

The goal of this experiment was to see whether the PNGase was active under strong reducing environment without any antioxidant moiety (GST tag).

Therefore, the inventors assessed 2 different reducing conditions with strong reducing reagents DTT and TCEP to test the activity of PNGase.

The resulting SDS-page gel is shown in FIG. 8. NC represents negative control where IgG was only reduced but with no deglycosylation taking place. Lanes NH1-NH6 represent the above-defined experimental conditions, namely NH1: TCEP 40 mM, NH2: TCEP 50 mM, NH3: TCEP 100 mM, NH4: TCEP 250 mM, NH5: TCEP 300 mM, NH6: DTT 50 mM.

From the gel, light bands in NH1-NH6 representing PNGase without GST tag can be observed with molecular weight at 60.8 kDa between 50 kDa and 75 kDa ladders, indicating the GST tag was successfully removed from the enzyme GST-PNGase.

Moreover, strong bands at 50 kDa position were observed representing the heavy chain of the IgG. Compared to the NC lane with negative control, no mass shift was observed in all the NH1-NH6 lanes indicating that, under those reducing environments, PNGase without GST tag was not active enough to enable deglycosylation to happen.

These results thus confirm the significance of the antioxidant moiety (GST tag) for PNGase to be enzymatically active under strong reducing environment.

### 5. Example 5: GST-PNGase is Active Under Strong Reducing Environment, which is not the Case of GST-PNGase F

Activity of GST-PNGase on Immunoglobulin G (IgG) under strong reducing environment was compared to activity of GST-PNGase F in the same conditions.

### 5.1. Experimental Conditions

Glycoprotein IgG was used as a deglycosylation target. It comprises a glycosylated heavy chain at around 50 kDa and a light chain of around 25 kDa without glycosylation. Deglycosylation of the IgG heavy chain was thus assessed.

The protein IgG I4505 was bought from Merck (Darmstadt, Germany). 10 μg of IgG was used for each condition.

IgG was first reduced under a different strong reducing environment: Tris(2-carboxyethyl)phosphine (TCEP) at 450 mM.

Deglycosylation assay was then performed under the optimal conditions for GST-PNGase (i.e., at 22° C. at pH=2.5) and GST-PNGase F (i.e., at 37° C. at pH=7.8) for overnight reaction on thermomixer at 750 rpm.

Deglycosylated products were analyzed with SDS-PAGE gel stained by Coomassie Blue.

### 5.2. Results

Results are presented in FIG. 9. NC represent the negative control in lanes 1 and 6. Commercial PNGase F alone is shown at lane 5 near 37 kDa position. Products of IgG heavy chain deglycosylation by GST-PNGase F are shown in lane 2 and products of IgG heavy chain deglycosylation by GST-PNGase are shown in lane 3.

Comparing these two results by looking at the tail of the two IgG heavy chain bands, a slight mass shift from lane 2 to 3 can be observed due to the glycan removal from the heavy chain by GST-PNGase.

On the contrary, the mass of the product of the heavy chain deglycosylation by GST-PNGase F in lane 2 is at the same level as the two negative controls, which shows that no deglycosylation happened using GST-PNGase-F under this strong reducing environment.

These results thus show that the Acidobacteria PNGase of the invention, fused to an antioxidant moiety, keeps a glycosylation activity, even under strong reducing environment (with TCEP 450 mM), which does not seem to be the case of non-Acidobacteria PNGase (such as PNGase-F) even if fused to an antioxidant moiety.

### BIBLIOGRAPHIC REFERENCES

Wang et al. 2014 (doi:10.1042/BSR20140148)
Guo et al., 2020 (doi: 10.3389/fbioe.2020.00741)
Uhlmann, E. & Peyman, A. (1990) Chemical Reviews, 90, 543-584
Schomburg D., Schomburg I., Springer Handbook of Enzymes. 2 edn. Heidelberg: Springer; 2001-2009
Liébecq C., IUPAC-IUBMB Joint Commission on Biochemical Nomenclature (JCBN) and Nomenclature Committee of IUBMB (NC-IUBMB) Biochem. Mol. Biol. Int. 1997; 43:1151-1156
IUBMB (1992), Enzyme Nomenclature 1992, Academic Press, San Diego
Schomburg D, Schomburg I. Methods Mol Biol. 2010; 609:113-28
Chang A, Schomburg I, Placzek 5, Jeske L, Ulbrich M, Xiao M, Sensen C W, Schomburg D, Nucleic Acids Res. 2015 January; 43. Epub 2014 Nov. 5. BRENDA in 2015: exciting developments in its 25th year of existence
Thomas D. Pollard, William C. Earnshaw, Jennifer Lippincott-Schwartz, Graham Johnson Cell Biology E-Book, Elsevier Health Sciences, 1 Nov. 2016
Mohammed Zourob, Recognition Receptors in Biosensors, DOI 10.1007/978-1-4419-0919-0, Springer-Verlag New York 2010
Abbas, Lichtman, Pillai, Cellular and Molecular Immunology E-Book, Elsevier Health Sciences, Aug. 22, 2014
Bayer V., An Overview of Monoclonal Antibodies. Semin Oncol Nurs. 2019 Sep. 2:150927; Wang W, Wang E Q, Balthasar J P. Monoclonal antibody pharmacokinetics and pharmacodynamics. Clin Pharmacol Ther. 2008 November; 84(5):548-58)
G. J. V. Nossal, G L Ada, Antigens, Lymphoid Cells and the Immune Response, Academic Press, 1971
Marc H. V. Van Regenmortel, Structure of Antigens, Volume 3 CRC Press, Dec. 20, 1995
Edouard Drouhet, Garry T. Cole, Louis De Repentigny, Jean Latge, Fungal Antigens: Isolation, Purification, and Detection, Springer Science t Business Media, Nov. 11, 2013
Graziano D. F., Finn O. J. (2005) Tumor Antigens and Tumor Antigen Discovery. In: Khleif S. N. (eds) Tumor Immunology and Cancer Vaccines. Cancer Treatment and Research, vol 123. Springer, Boston, MA
Wang M, Claesson M H, Methods Mol Biol. 2014; 1184:309-17. Classification of human leukocyte antigen (HLA) supertypes
Galperin, Fernindez-Suirez, Rigden, The 24th annual Nucleic Acids Research database issue: a look back and upcoming changes, NAR, Volume 45, Issue D1, January 2017, Pages D1-D11
Needleman and Wunsch (A general method applicable to the search for similarities in the amino acid sequence of two proteins, J. Mol. Biol., 1970, March; 48(3):443-453
Wada et al., 1992, Nucleic Acids Res. 20:2111
Nakamura et al., 1996, Nucleic Acids Res. 24:214
Sambrook et al. 2001, Molecular Cloning-A Laboratory Manual, Cold Spring Harbor Laboratory
Chartier et al. 1996, J. Virol. 70: 4805-10
Paul et al. 2002, Cancer gene Ther. 9: 470-7
Mercado-Pimentel et aL., 2002

1. A fusion polypeptide comprising a polypeptide having an N-glycosidase activity fused to an antioxidant moiety, wherein the polypeptide having an N-glycosidase activity is a polypeptide from a bacterium belonging to the Acidobacteria phylum.

2. The fusion polypeptide of claim 1, wherein the bacterium belongs to the Acidobacteriia class.

3. The fusion polypeptide of claim 2, wherein the bacterial belongs to the Terriglobus genus.

4. (canceled)

5. The fusion polypeptide of claim 1, wherein the antioxidant moiety is selected from the group consisting of antioxidant polypeptides, antioxidant provitamins, antioxidant vitamins, antioxidant flavonoids, compounds having a thiol group, compounds having a phenol group, polyphenols, carotenes, glutathione, lipoic acid, uric acid, tocopherols, and ubiquinol (coenzyme Q).

6. (canceled)

7. The fusion polypeptide of claim 1, wherein the antioxidant moiety is an antioxidant polypeptide selected from the group consisting of glutathione S-transferase, glutathione peroxidase, thioredoxin, catalase, and superoxide dismutase.

8. (canceled)

9. The fusion polypeptide of claim 1, wherein the antioxidant moiety is fused to the N-terminus of the polypeptide having an N-glycosidase activity, or to the C-terminus of the polypeptide having an N-glycosidase activity, or within the polypeptide having an N-glycosidase activity, or any combination thereof.

10. The fusion polypeptide of claim 1, wherein the antioxidant moiety is fused to the polypeptide having an N-glycosidase activity directly or through a linker.

11. The fusion polypeptide of claim 1, wherein the polypeptide having an N-glycosidase activity comprises an amino acid sequence having at least 85% identity with SEQ ID NO:1.

12. The fusion polypeptide of claim 1, wherein the polypeptide having an N-glycosidase activity comprises an amino acid sequence having at least 85% identity with SEQ ID NO:1 and is fused to an antioxidant polypeptide.

13. The fusion polypeptide of claim 11, wherein the polypeptide having an N-glycosidase activity comprises the amino acid sequence SEQ ID NO: 1.

14. A nucleic acid molecule encoding the fusion polypeptide according to claim 1, wherein the antioxidant moiety is an antioxidant polypeptide.

15. A vector comprising a nucleic acid molecule encoding the fusion polypeptide according to claim 1 wherein the antioxidant moiety is an antioxidant polypeptide, and wherein the vector is a plasmid or a viral vector.

16. A host cell comprising (a) a nucleic acid molecule encoding the fusion polypeptide according to claim 1 wherein the antioxidant moiety is an antioxidant polypeptide or (b) a vector comprising said nucleic acid molecule wherein the vector is a plasmid or a viral vector.

17. A composition comprising:
(i) the fusion polypeptide according to claim 1,
(ii) a nucleic acid molecule encoding the fusion polypeptide of claim 1 wherein the antioxidant moiety is an antioxidant polypeptide,
(iii) a vector comprising a nucleic acid molecule encoding the fusion polypeptide of claim 1 wherein the antioxidant moiety is an antioxidant polypeptide and wherein the vector is a plasmid or a viral vector,
(iv) a host cell comprising the nucleic acid molecule (ii) or the vector (iii), or
(v) any combination thereof.

18. The composition according to claim 17, wherein the fusion polypeptide, the nucleic acid molecule, the vector, the host cell, or any combination thereof is solubilized in a buffer comprising formic acid and/or glycine-HCl.

19. A method for purifying a fusion polypeptide as defined in claim 1, comprising the step of recovering the fusion polypeptide in a buffer comprising formic acid and/or glycine-HCl.

20. A method for producing a fusion polypeptide as defined in claim 1, comprising the steps of:
b) introducing a vector comprising a nucleic acid molecule encoding the fusion polypeptide of claim 1 wherein the antioxidant moiety is an antioxidant polypeptide and wherein the vector is a plasmid or a viral vector, in a host cell;
c) growing the host cell of step b) under suitable conditions so as to allow the production of the fusion polypeptide, and
d) optionally purifying the fusion polypeptide of step c) by recovering the fusion polypeptide in a buffer comprising formic acid and/or glycine-HCl.

21. (canceled)

22. A method for producing a fusion polypeptide as defined in claim 1, comprising the steps of:
b) introducing a vector comprising a nucleic acid molecule encoding a polypeptide having an N-glycosidase activity as defined in claim 1 in a host cell;
c) growing the host cell of step b) in the presence of an antioxidant moiety as defined in claim 1, under suitable conditions so as to allow the production of a polypeptide having an N-glycosidase activity and the fusion of the antioxidant moiety to the produced polypeptide having an N-glycosidase activity, and
d) optionally purifying the fusion polypeptide of step c) by recovering the fusion polypeptide in a buffer comprising formic acid.

23. (canceled)

24. (canceled)

25. A kit comprising:
a) a container comprising:
(i) the fusion polypeptide according to claim 1,
(ii) a nucleic acid molecule encoding the fusion polypeptide of claim 1 wherein the antioxidant moiety is an antioxidant polypeptide,
(iii) a vector comprising a nucleic acid molecule encoding the fusion polypeptide of claim 1 wherein the antioxidant moiety is an antioxidant polypeptide and wherein the vector is a plasmid or a viral vector,
(iv) a host cell comprising the nucleic acid molecule (ii) or the vector (ii), or
(v) any combination thereof;
b) optionally a different container comprising a buffer, a vehicle, an excipient, and any combination thereof; and
c) optionally instructions of use.


SEQUENCE LISTING (17 sequences)

SEQ ID NO: 1 - protein, 569 aa, Terriglobus roseus
MPRILCRPLSLQRCVLAASLTFAVTAAAQVVTVDTKPVVGSSNPATAEPPVTRPTSAPCTVTLFDNQVFKDYSAKPLTYAPPTGCRGHWAKVVLTVDFTVTAGRQFDRTASLYLGNANIFYGTTAEPRAALSPSWHVERDVTDLSALLKTSQSGQAIVYNVVDSTYTGIIYGTAKLLFYPADFRNPPAVVPDVVIPVSGTNSPYELDTTSSQLTTSVVAPRNTVKAYLDVIAQSQSSDEFWYLCAPNDVAAKLQSCPSTAFRETEITIDGKPAGVAPVYPWIYTGGIDPYLWSEPIVGVQTLNFKPYRVDLTPFAGVLSDGAPHTVGVSVYNATSYFLATANLLLYTDPFRAQTGGGLLENTLSLAPTPLVVEKLTTDASGTTTGTVTVDSARTFTVKGYVNSALGRVETTVEQKVNFNSAQTFNVGTNDIQNAVQTSTVSSKTTSQLGFLKETTDKQISFPLTLNYAYTVNNDGTSAQVVTSDQKDVETETKSINGLPIFGSSRNEVVHTTDTLNYSAAGALVSATPTSASATYKAEDTLGKCFSRQVTSTARAVTNVTDGAGCPVKR

SEQ ID NO: 2 - protein, 239 aa, synthetic construct
  source 1..239: note = glutathione S-transferase (GST)
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPNSRVDSSGRIVT

SEQ ID NO: 3 - protein, 203 aa, Homo sapiens
MCAARLAAAAAAAQSVYAFSARPLAGGEPVSLGSLRGKVLLIENVASLUGTTVRDYTQMNELQRRLGPRGLVVLGFPCNQFGHQENAKNEEILNSLKYVRPGGGFEPNFMLFEKCEVNGAGAHPLFAFLREALPAPSDDATALMTDPKLITWSPVCRNDVAWNFEKFLVGPDGVPLRRYSRRFQTIDIEPDIEALLSQGPSCA

SEQ ID NO: 4 - protein, 190 aa, Homo sapiens
MAFIAKSFYDLSAISLDGEKVDFNTFRGRAVLIENVASLUGTTTRDFTQLNELQCRFPRRLVVLGFPCNQFGHQENCQNEEILNSLKYVRPGGGYQPTFTLVQKCEVNGQNEHPVFAYLKDKLPYPYDDPFSLMTDPKLIIWSPVRRSDVAWNFEKFLIGPEGEPFRRYSRTFPTINIEPDIKRLLKVAI

SEQ ID NO: 5 - protein, 226 aa, Homo sapiens
MARLLQASCLLSLLLAGFVSQSRGQEKSKMDCHGGISGTIYEYGALTIDGEEYIPFKQYAGKYVLFVNVASYUGLTGQYIELNALQEELAPFGLVILGFPCNQFGKQEPGENSEILPTLKYVRPGGGFVPNFQLFEKGDVNGEKEQKFYTFLKNSCPPTSELLGTSDRLFWEPMKVHDIRWNFEKFLVGPDGIPIMRWHHRTTVSNVKMDILSYMRRQAALGVKRK

SEQ ID NO: 6 - protein, 197 aa, Homo sapiens
MSLGRLCRLLKPALLCGALAAPGLAGTMCASRDDWRCARSMHEFSAKDIDGHMVNLDKYRGFVCIVTNVASQUGKTEVNYTQLVDLHARYAECGLRILAFPCNQFGKQEPGSNEEIKEFAAGYNVKFDMFSKICVNGDDAHPLWKWMKIQPKGKGILGNAIKWNFTKFLIDKNGCVVKRYGPMEEPLVIEKDLPHYF

SEQ ID NO: 7 - protein, 221 aa, Homo sapiens
MTTQLRVVHLLPLLLACFVQTSPKQEKMKMDCHKDEKGTIYDYEAIALNKNEYVSFKQYVGKHILFVNVATYCGLTAQYPELNALQEELKPYGLVVLGFPCNQFGKQEPGDNKEILPGLKYVRPGGGFVPSFQLFEKGDVNGEKEQKVFSFLKHSCPHPSEILGTFKSISWDPVKVHDIRWNFEKFLVGPDGIPVMRWSHRATVSSVKTDILAYLKQFKTK

SEQ ID NO: 8 - protein, 221 aa, Homo sapiens
MFQQFQASCLVLFFLVGFAQQTLKPQNRKVDCNKGVTGTIYEYGALTLNGEEYIQFKQFAGKHVLFVNVAAYUGLAAQYPELNALQEELKNFGVIVLAFPCNQFGKQEPGTNSEILLGLKYVCPGSGFVPSFQLFEKGDVNGEKEQKVFTFLKNSCPPTSDLLGSSSQLFWEPMKVHDIRWNFEKFLVGPDGVPVMHWFHQAPVSTVKSDILEYLKQFNTH

SEQ ID NO: 9 - protein, 187 aa, Homo sapiens
MVAATVAAAWLLLWAAACAQQEQDFYDFKAVNIRGKLVSLEKYRGSVSLVVNVASECGFTDQHYRALQQLQRDLGPHHFNVLAFPCNQFGQQEPDSNKEIESFARRTYSVSFPMFSKIAVTGTGAHPAFKYLAQTSGKEPTWNFWKYLVAPDGKVVGAWDPTVSVEEVRPQITALVRKLILLKREDL

SEQ ID NO: 10 - protein, 209 aa, Homo sapiens
MEPLAAYPLKCSGPRAKVFAVLLSIVLCTVTLFLLQLKFLKPKINSFYAFEVKDAKGRTVSLEKYKGKVSLVVNVASDCQLTDRNYLGLKELHKEFGPSHFSVLAFPCNQFGESEPRPSKEVESFARKNYGVTFPIFHKIKILGSEGEPAFRFLVDSSKKEPRWNFWKYLVNPEGQVVKFWKPEEPIEVIRPDIAALVRQVIIKKKEDL

SEQ ID NO: 11 - protein, 105 aa, Homo sapiens
MVKQIESKTAFQEALDAAGDKLVVVDFSATWCGPCKMIKPFFHSLSEKYSNVIFLEVDVDDCQDVASECEVKCMPTFQFFKKGQKVGEFSGANKEKLEATINELV

SEQ ID NO: 12 - protein, 527 aa, Homo sapiens
MADSRDPASDQMQHWKEQRAAQKADVLTTGAGNPVGDKLNVITVGPRGPLLVQDVVFTDEMAHFDRERIPERVVHAKGAGAFGYFEVTHDITKYSKAKVFEHIGKKTPIAVRFSTVAGESGSADTVRDPRGFAVKFYTEDGNWDLVGNNTPIFFIRDPILFPSFIHSQKRNPQTHLKDPDMVWDFWSLRPESLHQVSFLFSDRGIPDGHRHMNGYGSHTFKLVNANGEAVYCKFHYKTDQGIKNLSVEDAARLSQEDPDYGIRDLFNAIATGKYPSWTFYIQVMTFNQAETFPFNPFDLTKVWPHKDYPLIPVGKLVLNRNPVNYFAEVEQIAFDPSNMPPGIEASPDKMLQGRLFAYPDTHRHRLGPNYLHIPVNCPYRARVANYQRDGPMCMQDNQGGAPNYYPNSFGAPEQQPSALEHSIQYSGEVRRFNTANDDNVTQVRAFYVNVLNEEQRKRLCENIAGHLKDAQIFIQKKAVKNFTEVHPDYGSHIQALLDKYNAEKPKNAIHTFVQSGSHLAAREKANL

SEQ ID NO: 13 - protein, 154 aa, Homo sapiens [sequence omitted]

SEQ ID NO: 14 - protein, 808 aa, synthetic construct
  source 1..808: note = N-glycosidase of Terriglobus roseus (strain DSM 18391) fused to GST
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPNSRVDSSGRIVTMPRILCRPLSLQRCVLAASLTFAVTAAAQVVTVDTKPVVGSSNPATAEPPVTRPTSAPCTVTLFDNQVFKDYSAKPLTYAPPTGCRGHWAKVVLTVDFTVTAGRQFDRTASLYLGNANIFYGTTAEPRAALSPSWHVERDVTDLSALLKTSQSGQAIVYNVVDSTYTGIIYGTAKLLFYPADFRNPPAVVPDVVIPVSGTNSPYELDTTSSQLTTSVVAPRNTVKAYLDVIAQSQSSDEFWYLCAPNDVAAKLQSCPSTAFRETEITIDGKPAGVAPVYPWIYTGGIDPYLWSEPIVGVQTLNFKPYRVDLTPFAGVLSDGAPHTVGVSVYNATSYFLATANLLLYTDPFRAQTGGGLLENTLSLAPTPLVVEKLTTDASGTTTGTVTVDSARTFTVKGYVNSALGRVETTVEQKVNFNSAQTFNVGTNDIQNAVQTSTVSSKTTSQLGFLKETTDKQISFPLTLNYAYTVNNDGTSAQVVTSDQKDVETETKSINGLPIFGSSRNEVVHTTDTLNYSAAGALVSATPTSASATYKAEDTLGKCFSRQVTSTARAVTNVTDGAGCPVKR

SEQ ID NO: 15 - DNA, 31 bp, synthetic construct
  source 1..31: mol_type = other DNA; note = primer
gaattctatgccccgcatcttgtgccgccct

SEQ ID NO: 16 - DNA, 30 bp, synthetic construct
  source 1..30: mol_type = other DNA; note = primer
atactcgaggcgtttcaccgggcagcctgc

SEQ ID NO: 17 - protein, 21 aa, synthetic construct
  source 1..21: note = peptide from Figure 3(continued)
YIAWPLQGWQATFGGGDHPPK